(19) United States
(12) Patent Application Publication
(10) Pub. No.: US 2025/0093360 A1
(43) Pub. Date: Mar. 20, 2025

(54) DNA BASED SENSORS

(71) Applicant: George Mason University, Fairfax, VA (US)

(72) Inventors: Remi Veneziano, Falls Church, VA (US); Parag V. Chitnis, Burke, VA (US); Giovanni Giammanco, Hanover, MD (US); John R. Cressman, Fairfax, VA (US)

(21) Appl. No.: 18/775,495

(22) Filed: Jul. 17, 2024

Related U.S. Application Data
(60) Provisional application No. 63/583,317, filed on Sep. 18, 2023.

(51) Int. Cl. G01N 33/58; G01N 21/64

(52) U.S. Cl. CPC G01N 33/587; G01N 21/6428; G01N 21/6456; G01N 2021/6439; G01N 2458/30

(57) ABSTRACT

Provided is a nanoparticle comprising: a DNA scaffold comprising an attached dye, an attached functional moiety, or a combination thereof. In some embodiments, the DNA scaffold comprises a plurality of DNA oligonucleotides, wherein each oligonucleotide is independently optionally attached to a dye and/or a functional moiety. Provided is a method of cell voltage sensing at a target site comprising providing the nanoparticle of any one of the preceding embodiments at the target site; and monitoring cell voltage at the target site. Also provided is a method of fluorescence or absorbance imaging at a target site comprising providing the nanoparticle of any one of the preceding embodiments at the target site; and monitoring fluorescence or absorbance at the target site.

### CROSS-REFERENCE TO RELATED APPLICATIONS

The instant application is entitled to priority under 35 U.S.C. § 119(e) to U.S. Provisional Application No. 63/583,317 filed Sep. 18, 2023, which is hereby incorporated by reference in its entirety.

### GOVERNMENT INTEREST

This invention was made with government support under grant number 2128821 awarded by the National Science Foundation. The government has certain rights in the invention.

### FIELD

The general inventive concepts relate to the field of DNA-based near infrared sensors.

### SEQUENCE LISTING

The content of the electronic sequence listing (GMU-24-007-306564-00044.xml; Size: 65,611 bytes; and Date of Creation: Jul. 16, 2024) is herein incorporated by reference in its entirety.

### BACKGROUND

While the Indocyanine Green (ICG) dye has become a standard in fluorescence imaging in research, (1) surgical navigation, (2, 3) and medical diagnostics, (4) ICG is still an emerging probe in many other fluorescence imaging applications including voltage sensing. (5) Indeed, even though ICG is a FDA approved near infrared (NIR) absorbing dye with voltage sensing capabilities, (6) it offers less than ideal optical characteristics that limits its uses. For instance, ICG is highly susceptible to photobleaching, lacks inherent targeting capability, and has variable light characteristics. (7, 8) The dye's peak absorbance depends on its concentration, shifting between two peaks one at higher concentrations (1000 μM) at ˜690 nm and one at lower concentrations (10 μM) at ˜790 nm. (9) At lower concentrations, ICG favors its monomeric form. However, as its concentration increases, oligomerization is favored. (10) When using ICG in vivo, not only does the absorbance shift with concentration, but also fluorescence intensity changes with time and environment. (10-13) These factors all contribute to an overall inconsistent and non-uniform fluorescence intensity. Gold colloids have even been used to stabilize light characteristics and provide targeting abilities to ICG in retinal angiography. (14) There remains a need for a robust, biocompatible NIR voltage sensor with targeting capabilities for in vivo applications.

### SUMMARY

Provided is a nanoparticle comprising: a DNA scaffold comprising an attached dye, an attached functional moiety, or a combination thereof.

In some embodiments, the functional moiety is not a therapeutic drug.

In some embodiments, the DNA scaffold comprises a plurality of DNA oligonucleotides, wherein each oligonucleotide is independently optionally attached to a dye and/or a functional moiety.

In some embodiments, the DNA scaffold comprises one or more oligonucleotides of SEQ ID NO: 1 to 63, or an oligonucleotide having 95%, 96%, 97%, 98%, 99% sequence identity thereto. In some embodiments, the DNA scaffold comprises one or more oligonucleotide selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, or an oligonucleotide having 95%, 96%, 97%, 98%, 99% sequence identity thereto. In some embodiments, the DNA scaffold comprises an oligonucleotide listed in Table 1, Table 2, Table 3, Table 4, Table 5, Table 6, Table 7, or Table 8, or an oligonucleotide having 95%, 96%, 97%, 98%, 99% sequence identity thereto.

In some embodiments, the DNA scaffold comprises one or more arms. In some embodiments, the DNA scaffold comprises 1, 2, 3, 4, 5, 6, 7, 8, 9, or 10 arms.

In some embodiments, the DNA scaffold comprises one or more two dimensional (2D) or three dimensional (3D) structures. In some embodiments, the 2D structure is a wheel, a plate, or a ring. In further embodiments, the 3D structure is an octahedra, an icosahedra, a cube, a sphere, or any regular polyhedron.

In some embodiments, the DNA scaffold is symmetrical. In some embodiments, the DNA scaffold is asymmetrical.

In some embodiments, the dye is a cyanine dye.

In some embodiments, the dye is selected from the group consisting of indocyanine green (ICG), ICG-Xtra-OSu, IGC-Osu, IGC-Sulfo-Osu, IGC-PEG12-Osu, ICG-NHS, ICG-NH₂, pseudoisocyanine, merocyanine, fluorescein, tetramethylrhodamine (TAMRA), bis(2,4,6-trihydroxyphenyl)squaraine, tetrtakis(4-sulfonatophenyl)-porphyrin, antimony(III)-phthalocyanine, copper phthalocyanine, perylene bismide, hypericin, subphtalocyanine, heptamethyne cyanine, and combinations thereof.

In some embodiments, the functional moiety is selected from the group consisting of a steroid, a cell-penetrating peptide, a targeting peptide, an antibody, or a small molecule. In further embodiments, the functional moiety is a therapeutic drug or antibody. In further embodiments, the steroid is cholesterol.

In some embodiments, the functional moiety is not a therapeutic drug.

In some embodiments, the dye is attached to the oligonucleotide directly or via a linker.

In some embodiments, the functional moiety is attached to the oligonucleotide directly or via a linker.

In some embodiments, orientation of any attached dyes and/or functional moieties is designed. In some embodiments, geometry, orientation, and stoichiometry of any attached dyes and/or functional moieties is designed. In further embodiments, any attached dyes and/or functional moieties face the same direction on the oligonucleotide.

In some embodiments, the nanoparticle may be used as a sensor for bioelectric activity.

In further embodiments, the nanoparticle may be used as a cell voltage sensor.

In some embodiments, the nanoparticle may be used as a photoacoustic probe.

Provided is a nanoparticle produced by a process comprising mixing a plurality of DNA oligonucleotides to assemble a DNA scaffold, wherein each oligonucleotide is optionally attached to a dye and/or a functional moiety.

In some embodiments, orientation of any attached dyes and/or functional moieties is designed.

Provided is a method of cell voltage sensing at a target site comprising providing the nanoparticle of any one of the preceding embodiments at the target site; and monitoring cell voltage at the target site.

Provided is a method of cell voltage sensing at a target site comprising providing the nanoparticle produced by a process as described herein at the target site; and monitoring cell voltage at the target site.

Provided is a method of fluorescence imaging at a target site comprising providing the nanoparticle of any one of the preceding embodiments at the target site; and monitoring fluorescence or absorbance at the target site.

In some embodiments, imaging comprises NIR I, NIR II or photoacoustic imaging.

Provided is a method of fluorescence imaging at a target site comprising providing the nanoparticle produced by a process as described herein at the target site; and monitoring fluorescence or absorbance at the target site.

In some embodiments, imaging comprises NIR I, NIR II or photoacoustic imaging.

Provided is a method of absorbance or photoacoustic imaging at a target site comprising providing the nanoparticle of any one of the preceding embodiments at the target site; and monitoring absorbance or photoacoustic signal at the target site.

In some embodiments, imaging comprises NIR I, NIR II or photoacoustic imaging.

Provided is a method of absorbance or photoacoustic imaging at a target site comprising providing the nanoparticle produced by a process as described herein at the target site; and monitoring absorbance or photoacoustic signal at the target site.

In some embodiments, imaging comprises NIR I, NIR II or photoacoustic imaging.

### BRIEF DESCRIPTION OF THE FIGURES

FIGS. 1A-1C illustrate an exemplary novel DNA integrated voltage indicating nanoparticle (DIVIN) nanosensor. DIVIN is an exemplary embodiment of a DNA-dye nanoparticle (for example, DNA-ICG) that is used for voltage sensing. FIG. 1A is a schematic of a DNA scaffold visualized in Chimera that is shown with attached ICG (circles) and cholesterol (trihexagons) moieties. Dye and cholesterol groups are not to scale and their orientation and angle from the DIVIN surface are representative only to show that the structure has been designed to have different moieties facing the same direction. Scale bar: 2 nm. FIG. 1B is a schematic of the SPR experiments performed with the DIVIN probes to demonstrate binding to lipid membrane. A hydrophobic SPR sensor (left) is used to create a lipid monolayer from sub 100 nm liposomes (center) and the nanoparticles with or without cholesterol moieties are injected and their binding monitored (right). FIG. 1C shows representative SPR binding curves for the lipid monolayer formation (left) and for the binding of DIVIN-Chol or DIVIN probes.

FIGS. 2A-2C show the optical characterization and stability of DIVIN and ICG. FIG. 2A shows absorbance spectra of DIVIN compared to ICG at the concentrations: 120 βM, 60 μM, 20 μM, 10 μM, and 5 μM. FIG. 2B shows fluorescence spectra of peak fluorescence region about 780-840 nm for ICG and DIVIN at 120 μM, 60 μM, 20 μM, 10 μM, and 5 μM. FIG. 2C shows the stability of the absorbance spectra of DIVIN and ICG over a period of 72 hours.

FIGS. 3A-3B show DIVIN's stability in physiological conditions. FIG. 3A shows stability of the DIVIN probe in 20% serum measured with fluorescence resonance energy transfer (FRET). FIG. 3B shows the stability of the normalized photoacoustic (PA) signal intensity of DIVIN mixed in whole blood (solid) and in PBS (dashed) for a period of 75 min.

FIGS. 4A-4C show voltage sensing of DIVIN on labeled HeLa cell culture using patch-clamp fluorescence microscopy. FIG. 4A shows a schematic of the patch-clamp fluorescence microscope. Also shown is an inlet of a microscopy image of the cell culture that shows the electrode patched with the cell of interest. FIG. 4B is a plot of the relative change in fluorescence of two representative traces of two representative cells of interest (circle and diamond data points) over time (y-axis appears in black). The plot also shows the representative cells' potential as set by the patch-clamp (long dashes lines corresponding to the circle trace and short dashes lines corresponding to the diamond trace, the y-axis appears in black on the right). FIG. 4C is a plot of the percent change in the fluorescence signal (Δf/fₒ) of DIVIN versus the voltage across the cell membrane as set by the patch-clamp. The regression line can be seen in dashes, which is plotted over the measured value (rectangles and error bars). The regression was tested using a reduced Chi-squared test. The Chi-squared value is 1 for 6 measured points with an n≥5. Reduced χ²=1. Intercept: −0.24±0.15. Slope: −0.0147±0.0021. The data used to produce the plot were averaged from at least five replicates at every holding potential and acquired from at least two individual cells.

FIGS. 5A-5B show representative electrophoresis results of synthesis and stability of DIVIN. FIG. 5A shows electrophoresis results with three lanes. The first lane is the standard ladder, the second lane is the DIVIN probe at 0.5 μM, and the third lane is the DIVIN probe at 1 μM. FIG. 5B shows electrophoresis gel results with five lanes. The first lane is the standard ladder, the second lande is the DIVIN probe in PBS, the third lane is the DIVIN probe after one hour in PBS with 20% mouse serum, the fourth lane is the DIVIN probe after three hours in PBS with 20% mouse serum, the fifth lane is the DIVIN probe after six hours in PBS with 20% mouse serum. These experiments have been performed at least twice.

FIG. 6 shows a size measurement using dynamic light scattering (DLS) of DIVIN. (n=3 replicates)

FIG. 7 left panel shows Hela cell culture stained with DIVN-Chol. Right panel shows Hela culture stained with DIVIN probes lacking a cholesterol moiety.

FIG. 8 shows an optical characterization of DIVIN probe and free ICG. Representative absorbance spectra of DIVIN probes compared to ICG at five different concentrations: 120 μM, 60 μM, 20 μM, 10 μM, and 5 μM and normalized to the monomer peak (780 nm for ICG and 790 nm for DIVIN probe).

FIG. 9 shows representative electrophoresis results of the stability of DIVIN after 15 minutes of sonication. (N=2 replicates).

FIG. 10 shows a cell proliferation assay of HeLa cells stained with DIVIN in MTT. This plot shows the viability of HeLa cells across ascending concentrations of DIVIN from 1-20 μM (4 to 80 mM effective ICG concentration). Two-tailed significance values against the control are 0.002, 0.004, 0.003, in order of ascending concentration. (N=5 replicates)

FIG. 11 shows fluorescence pictures of DIVIN stained HeLa cells. Three separate HeLa cell cultures stained with DIVIN. The rectangles highlight the cell of interest which is continuous with the patch clamp electrode or the “patched” cell.

FIG. 12 shows representative FRET curves at different time points.

FIG. 13 shows a change in fluorescence intensity HeLa cells stain with Fluovolt. Plot of the relative change in fluorescence of one trace of one representative cell of interest (continuous line, y-axis on the left) over time. The plot also shows the representative cells' potential as set by the patch-clamp using the voltage clamp method (dashed lines, y-axis on the right).

FIGS. 14A-14F show data acquisition and analysis of fluorescence responses. FIG. 14A shows a fluorescent image of DIVIN labeled probes for a cell patched with a recording electrode.

FIG. 14B displays the normalized fluorescent signals for the regions of interest denoted by the rectangles in FIG. 14A, continuous for patched cell and dashed for the reference cell. FIG. 14C is the ratio of the fluorescent signal for the patched cell versus the reference cell. FIG. 14D displays the ratio from FIG. 14C after normalization and detrending. The detrending is performed by high-pass filtering of the data above 0.1 Hz, and is displayed in FIG. 14D and again in FIG. 14E along with the holding potential throughout the run. The fluorescent response to the voltage protocol, repeated five times, was averaged and is displayed in FIG. 14F; the holding voltage applied to the cell is displayed on the right axis.

FIGS. 15A-15C show the fluorescence response of DIVIN following depolarization of brain tissue with potassium ions. FIG. 15A displays a coronal brain slice from a mouse held in a mesh netting; a region of interest is denoted. FIG. 15B displays the fluorescence intensity change recorded within the region of interest as 30 mM KCl solution is added to the dish at approximately 25 seconds. FIG. 15C displays a time series montage of (enhanced) fluorescence response in the brain tissue following depolarization.

FIGS. 16A-16F show characterizations of the 1-arm and 5-arm DNA-ICG nanoparticles (NPs) in comparison with plain ICG dye. FIG. 16A shows 1-arm and 5-arm NP structures. Optical properties (FIG. 16B: Absorption measurements, FIG. 16C: Fluorescence intensity measurements), PA signals in PBS (FIG. 16D), PA signals and stability in blood (FIG. 16E), and normalized PA signals in blood (FIG. 16F) for ICG (Top Left), 1-arm DNA-ICG (Top Right) and 5-arm DNA-ICG (Bottom Center). wb: whole blood; bp: 50% blood and 50% PBS. DNA-ICG NPs exhibit stronger NIR absorption and PA signal intensity than ICG alone, enabling deeper tissue imaging. Concentration-dependence of PA signal of all probes was conserved when nano-probes were introduced into blood, similar to that observed when the probes were diluted in PBS. The fluorescence intensity at the peak wavelength was also increased for both nanoparticles compared to ICG.

FIGS. 17A-17D illustrate the DNA-ICG platform deployed in living systems and imaged with NIR-II. FIG. 17A: Schematic showing the spatial configuration of dyes (circles) and targeting moieties (trihexagons) to the DNA scaffold. FIG. 17B: Top: HeLa cells stained with the DNA-ICG platform lacking targeting moiety, Bottom: HeLa cells stained with DNA-ICG conjugated with targeting moiety. FIG. 17C: Left: mouse injected with free ICG dye, Center and Right: mice injected with DNA-ICG variants. FIG. 17D: Mouse treated with DNA-ICG after three hours; the circle of Willis (circled) visible beneath the skull.

FIGS. 18A-18B show four variants of the DNA-ICG NPs that were deployed in a capillary phantom model beneath tissue for NIR-II fluorescence imaging. FIG. 18A left panel: Schematic representations of DNA nanoparticle variants each conjugated with increasing quantities of ICG dye monomers (circles) (the numbers below each schematic represent the number of dyes per construct). FIG. 18A right panel: NIR-II fluorescence image of DNA-ICG NPs and free ICG dye in a capillary phantom obscured by tissue of various thickness. FIG. 18B: NIR-II fluorescence intensity along the cross-section for the different constructs and for different thickness of tissue (dotted line of FIG. 18A right panel). Fluorescence intensity was acquired for increasing tissue thicknesses.

FIG. 19 shows NIR-II fluorescence imaging of DNA-ICG NPs in the mouse model. Left: Whole body NIR-II fluorescence image of mouse injected with DNA-ICG NPs. A region of interest is denoted (square). Center: A blood vessel is isolated within the region of interest (bar). Right: The normalized fluorescence intensity of the isolated blood vessel cross section.

FIG. 20 shows short-term biodistribution of DNA-ICG NPs and free ICG dye in the mouse model. Left: Free ICG dye injected via tail vein. Right: DNA-ICG NPs injected via tail vein. The results show contrast in the lungs 0-12 seconds post-injection, in the vasculature 10-60 seconds post-injection, and in the liver after 1-minute post-injection.

FIG. 21 shows long-term biodistribution of DNA-ICG NPs and free ICG dye in the mouse model. Left: Free ICG dye injected via tail vein. Right: DNA-ICG NPs injected via tail vein. NIR-II fluorescence images show contrast in the liver 1-hour post-injection, in the intestines 3 hours post-injection, and the spleen and cecum 24 hours post-injection for both free ICG dye and DNA-ICG NPs.

FIG. 22 shows photostability time series of DNA-ICG NPs under continuous and intermittent excitation. Stability is conserved under intermittent excitation for nominal fluorescence imaging acquisition (continuous line). Photobleaching was observed under continuous, high intensity excitation over time (dashed line).

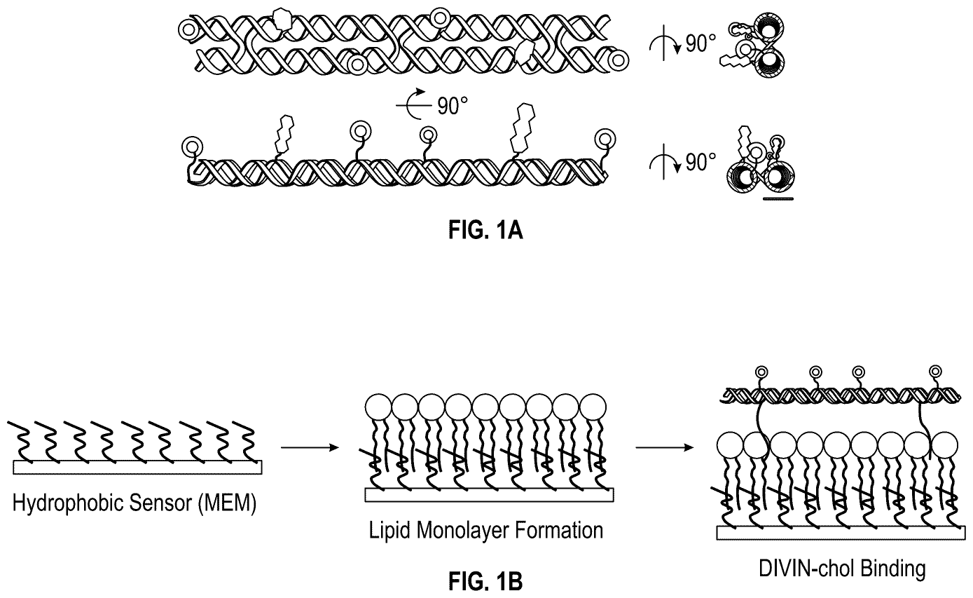

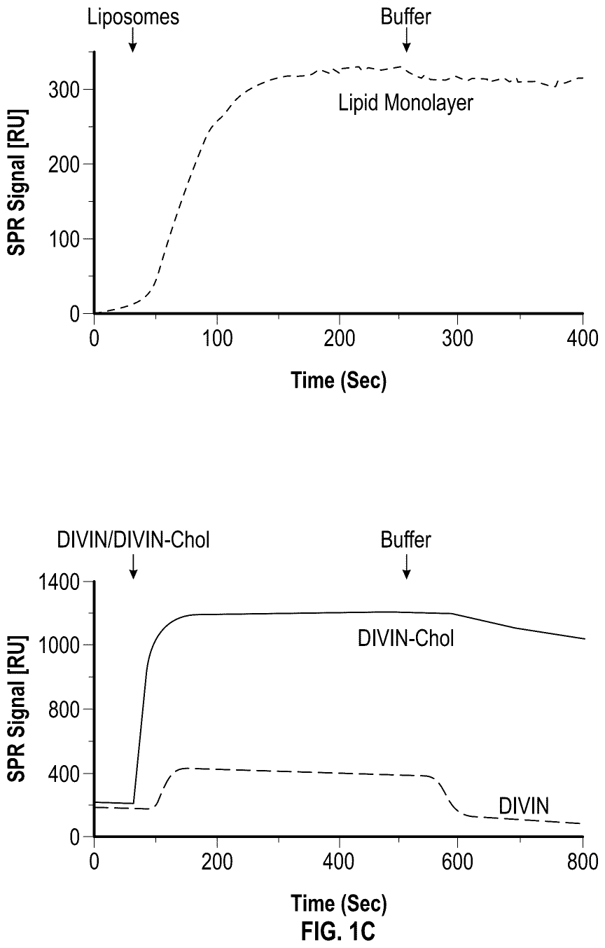

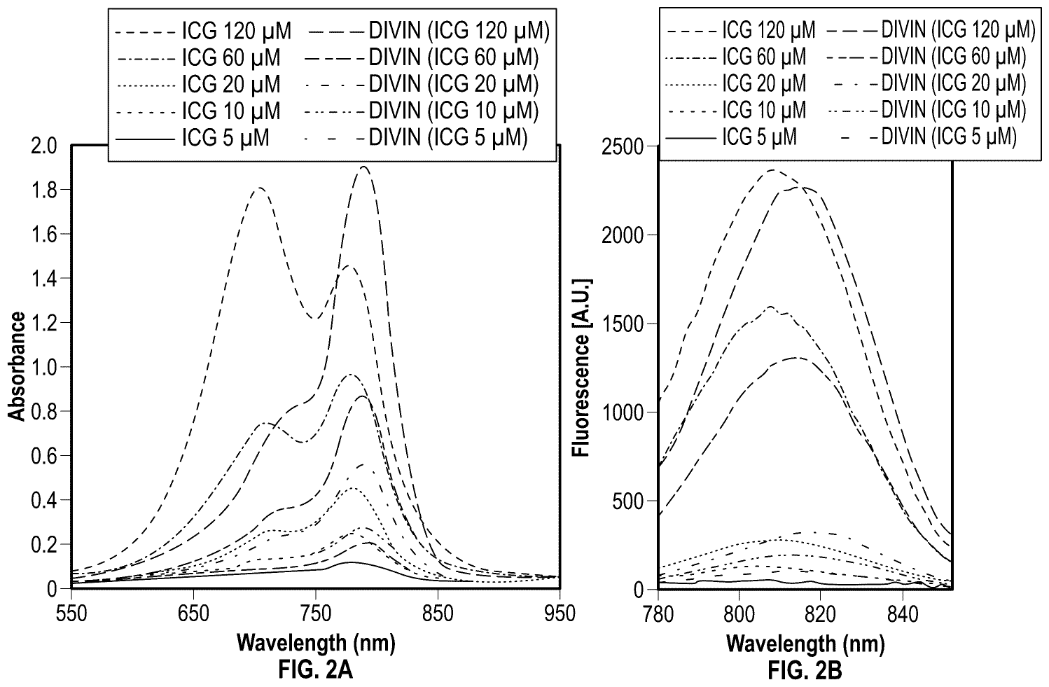

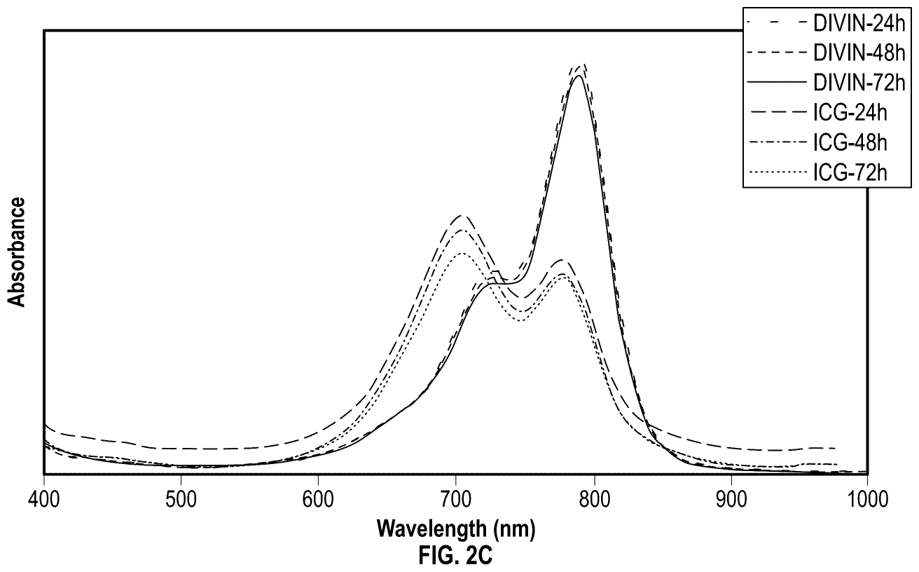

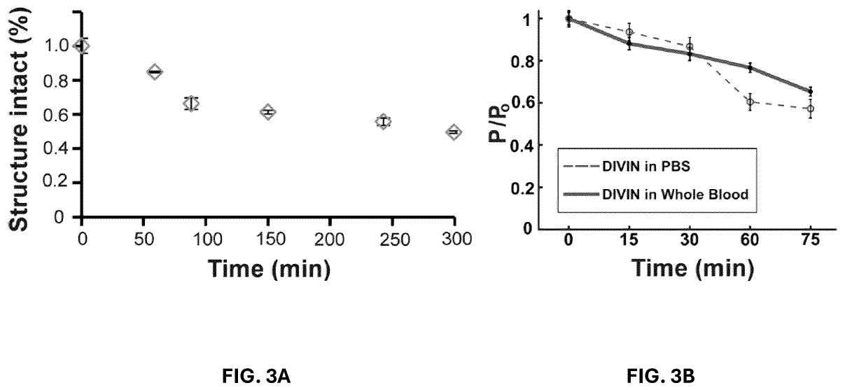

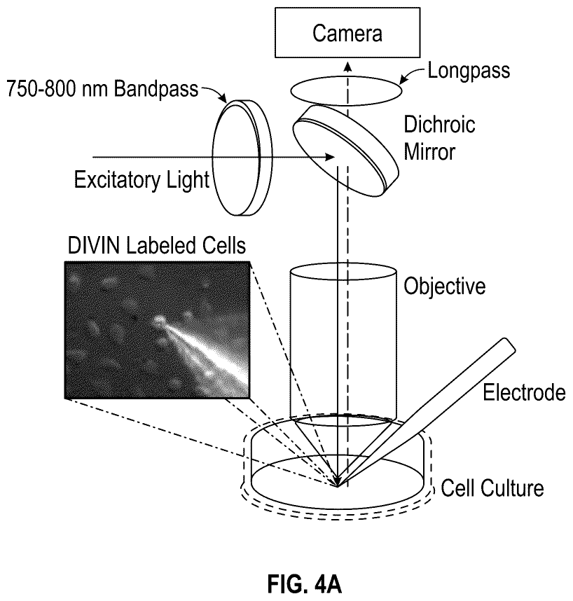

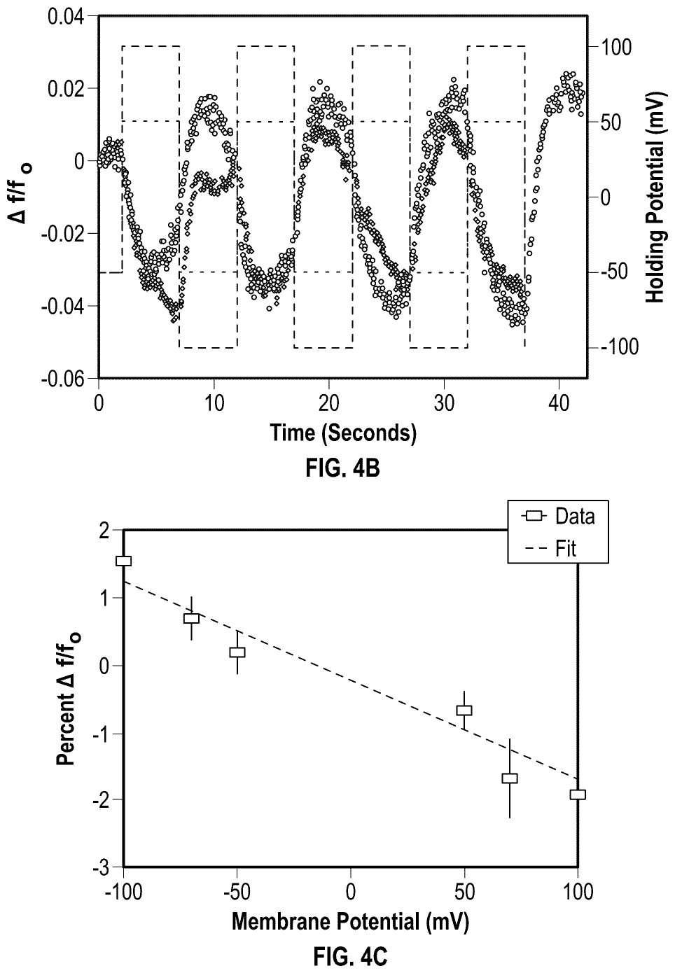

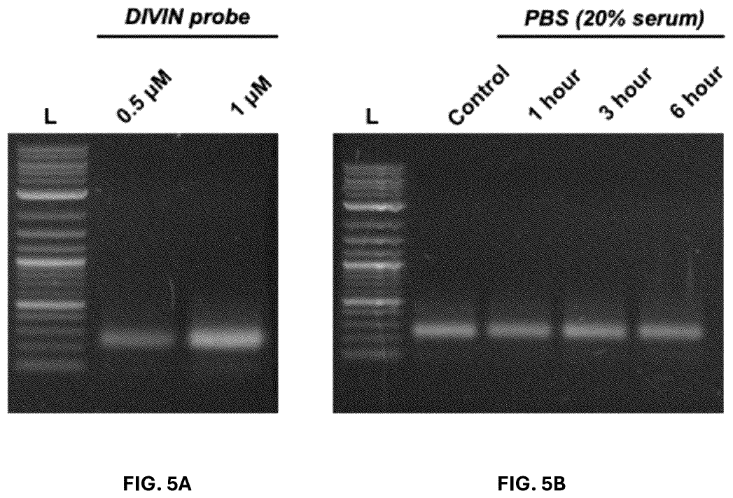

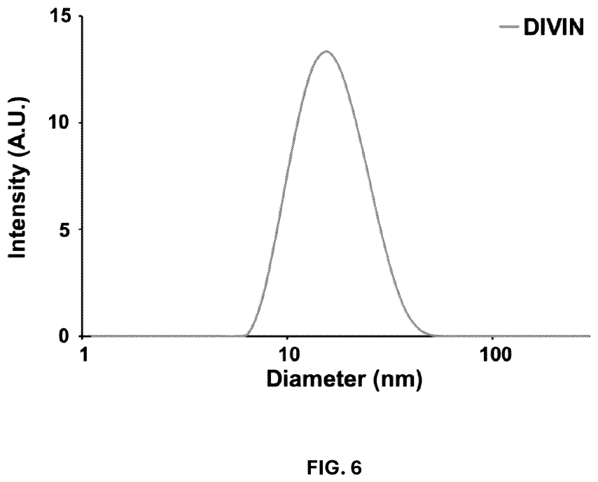

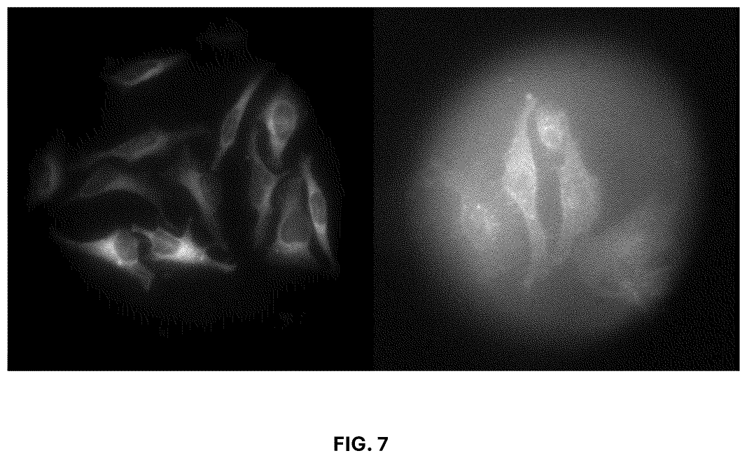

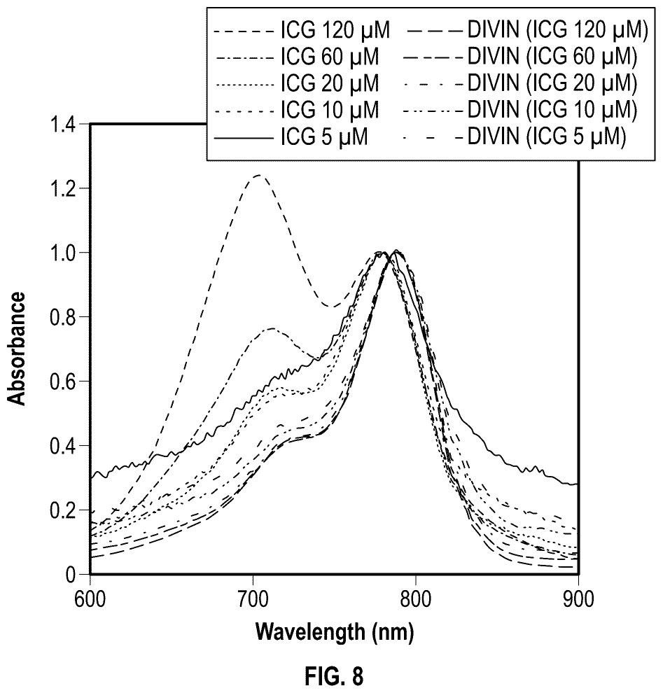

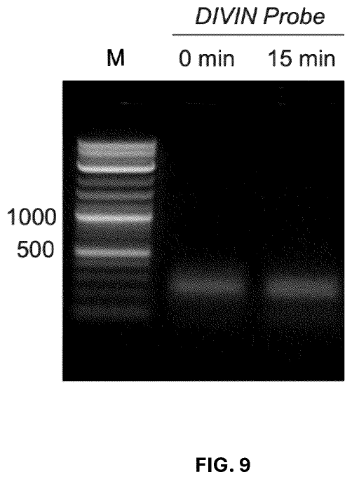

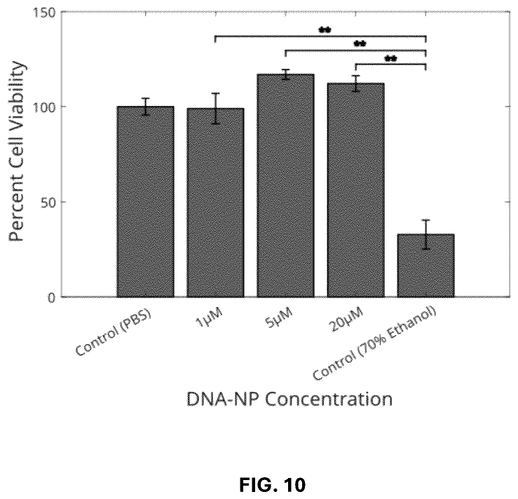

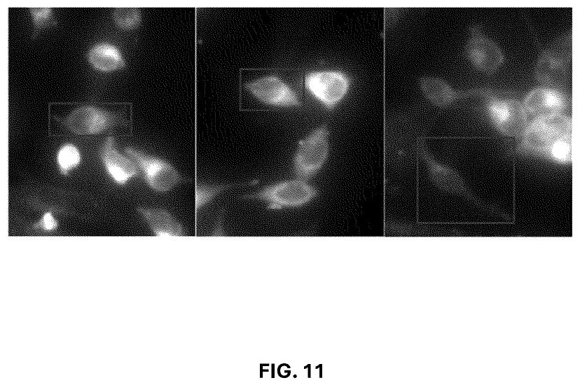

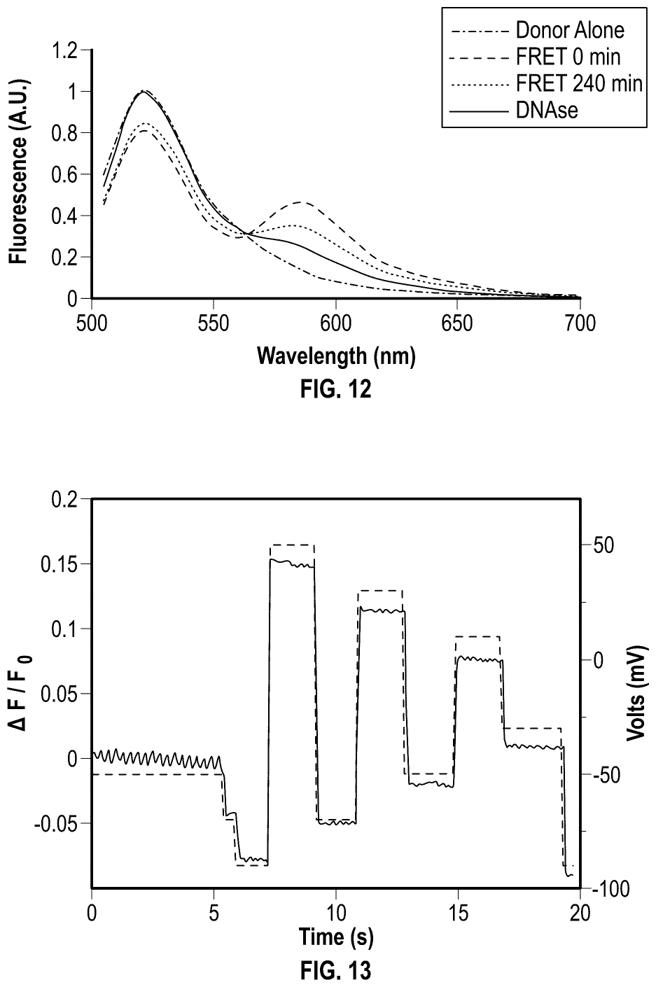

### DETAILED DESCRIPTION

While the general inventive concepts are susceptible of embodiment in many forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

It is also to be understood that the terminology used herein is for the purpose of describing particular embodiments only, and is not intended to be limiting.

The articles “a” and “an” are used herein to refer to one or more than one (i.e., to at least one) of the grammatical object of the article. By way of example, “a cell” means one cell or more than one cell.

“About” as used herein when referring to a measurable value such as an amount, a temporal duration, and the like, is meant to encompass variations of ±5%, preferably ±1%, and still more preferably ±0.1% from the specified value, as such variations are appropriate to perform the disclosed methods.

As used herein, the term “nanoscale” means particles having a size range between approximately 1 and 100 nanometers (nm).

As used herein, the term “nanometer” means 1/1,000,000,000 meter (m).

As used herein, the term “sub-micronscale” means particles having a size less than a micron (μm) and larger than 100 nm.

As used herein, the term “micron” means 1/1,000,000 meter (m).

As used herein, the term “microscale” means having a size of approximately 1 to 5 microns (μm).

In some embodiments of any of the compositions or methods described herein, a range is intended to comprise every integer or fraction or value within the range.

Embodiments described herein as “comprising” one or more features may also be considered as disclosure of the corresponding embodiments “consisting of” and/or “consisting essentially of” such features.

The inventors of the present application sought to mitigate some of ICGs drawbacks through the conjugation of ICG monomers to a DNA-based scaffold that could improve the optical properties of ICG while providing targeting abilities and stability.

This platform allows for unprecedented binding specificity and effective delivery of various payloads. The DNA scaffold can be designed and synthesized such that multiple desired functional and targeting moieties are placed in a designer geometry, orientation, and stoichiometry. This can provide a localized and controlled concentration to isolate ICG monomers. DNA nanotechnology has enabled the synthesis of complex molecular architectures with high structural fidelity using precise assembly of multiple single-stranded DNA via complementary Watson-Crick base pairing. These self-assembled DNA nanoparticles (DNA-NPs) are inherently biocompatible and can be programmed to accommodate precise organization of organic and inorganic molecules, which make them of high interest for several biomedical applications. Moreover, the DNA-NPs provide structural stability in physiological conditions and allow for bioconjugation to functional or targeting moieties of various nature. These DNA-NPs can be used to develop versatile nanosensors for multiple applications such as: infectious disease detection, cancer imaging, and functional brain imaging. There are few biocompatible NIR voltage sensors. Functionalization of established voltage sensors to DNA scaffolds may enhance the current range of voltage sensors for in vivo applications. Many fluorescent voltage-sensing probes designed for in vitro electrophysiology have relatively short excitation wavelengths in the UV-VIS range. In addition, many fluorescent voltage sensing probes have difficult targeting chemistries or require additional nanocarriers to provide biocompatibility and targeting abilities. With the higher tissue penetration depth and endogenous contrast absorption at a local minimum, biocompatible NIR voltage sensors could provide electrical activity monitoring without genetic manipulation to in vivo mouse models. Many current voltage indictors in vivo are genetically encoded voltage indicators (GEVIs), which are limited in their dynamic range due to saturation. Problems with inadequate expression, uneven expression, and limited localization limit GEVIs to application in a narrow range cell and tissue types.

There remains a need for a robust, biocompatible NIR voltage sensor with targeting capabilities for in vivo applications.

### Nanoparticles

Provided is a nanoparticle comprising: a DNA scaffold comprising an attached dye, an attached functional moiety, or a combination thereof.

In some embodiments, the functional moiety is not a therapeutic drug.

### DNA Scaffold

In some embodiments, the DNA scaffold comprises a plurality of DNA oligonucleotides, wherein each oligonucleotide is independently optionally attached to a dye and/or a functional moiety.

In some embodiments, the DNA scaffold comprises one or more oligonucleotides of SEQ ID NO: 1 to 63, or an oligonucleotide having 95%, 96%, 97%, 98%, or 99% sequence identity thereto. In some embodiments, the DNA scaffold comprises one or more oligonucleotide selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, or an oligonucleotide having 95%, 96%, 97%, 98%, 99% sequence identity thereto. In some embodiments, the DNA scaffold comprises an oligonucleotide listed in Table 1, Table 2, Table 3, Table 4, Table 5, Table 6, Table 7, or Table 8, or an oligonucleotide having 95%, 96%, 97%, 98%, or 99% sequence identity thereto.

In some embodiments, the DNA scaffold comprises one or more arms.

In some embodiments, the DNA scaffold has one arm and comprises the oligonucleotides listed in Tables 1 and 2, or oligonucleotides having 95%, 96%, 97%, 98%, or 99% sequence identity thereto.

In some embodiments, the DNA scaffold has 3 arms and comprises the oligonucleotides listed in Tables 3 and 4, or oligonucleotides having 95%, 96%, 97%, 98%, or 99% sequence identity thereto.

In some embodiments, the DNA scaffold has 4 arms and comprises the oligonucleotides listed in Tables 5 and 6, or oligonucleotides having 95%, 96%, 97%, 98%, or 99% sequence identity thereto.

In some embodiments, the DNA scaffold has 5 arms and comprises the oligonucleotides listed in Tables 7 and 8, or oligonucleotides having 95%, 96%, 97%, 98%, or 99% sequence identity thereto.

In some embodiments, the DNA scaffold comprises one or more two dimensional (2D) or three dimensional (3D) structures. In some embodiments, the 2D structure is a wheel, a plate, or a ring. In further embodiments, the 3D structure is an octahedra, an icosahedra, a cube, a sphere, or any regular polyhedron.

The DNA scaffold may be designed using software such as Tiamat. In some embodiments, the sequences of the different strands are designed to be highly orthogonal to maximize folding efficiency and avoid mispairing.

### Dyes and Functional Moieties

In some embodiments, the dye is a cyanine dye.

In some embodiments, the dye is selected from the group consisting of indocyanine green (ICG), ICG-Xtra-OSu, IGC-Osu, IGC-Sulfo-Osu, IGC-PEG12-Osu, ICG-NHS, ICG-NH₂, pseudoisocyanine, merocyanine, fluorescein, TAMRA, bis(2,4,6-trihydroxyphenyl)squaraine, tetrtakis(4-sulfonatophenyl)-porphyrin, antimony(III)-phthalocyanine, copper phthalocyanine, perylene bismide, hypericin, subphtalocyanine, heptamethyne cyanine, and combinations thereof.

In some embodiments, the functional moiety is selected from the group consisting of a steroid, a cell-penetrating peptide, a targeting peptide, an antibody, or a small molecule. In further embodiments, the functional moiety is a therapeutic drug or antibody. In some embodiments, the functional moiety is a steroid for targeting cell membrane, a cell-penetrating peptide for increased uptake by cells, a targeting peptide for specific cell targeting (e.g., RGD peptide for targeting integrin receptors), a protein for cell membrane receptors and or extra cellular matrix protein targeting (e.g., antibodies for targeting collagen), a small molecule, a drug, or an antibody for drug delivery (theranostic modality). In further embodiments, the steroid is cholesterol.

In some embodiments, the functional moiety is not a therapeutic drug.

In some embodiments, the dye is attached to the oligonucleotide directly or via a linker. In some embodiments, amino modified oligonucleotides are conjugated with ICG fluorophore by incubating them with ICG-Xtra-Osu, IGC-Osu, IGC-Sulfo-Osu or IGC-PEG12-Osu. In further embodiments, amino modified oligonucleotides are conjugated with ICG fluorophore by incubating them with ICG-Xtra-Osu. IGC, ICG-Xtra-Osu, IGC-Osu, IGC-Sulfo-Osu or IGC-PEG12-Osu are commercially available, for example from AAT Bioquest (Pleasanton, CA, USA). Thiol-modified oligonucleotides can be modified with ICG-thiol or ICG maleimide commercially available from Diagnocine and AAT bioquest, respectively. DBCO-modified oligonucleotides can be modified with azide-modified ICG commercially available from AAT Bioquest.

In some embodiments, the dye has been functionalized with an azide group. In further embodiments, the dye is attached to the nucleotide by employing copper-free click chemistry with the azide group. The oligonucleotides are modified with DBCO group and incubated with the azide modified ICG dye.

In some embodiments, the functional moiety is attached to the oligonucleotide directly or via a linker. In further embodiments, the linker comprises a polyethylene glycol (PEG) group of various length such as but not limited to 1000 Da, 2000 Da, 3000 Da, 5000 Da, 10000 Da. The PEG linker can be attached to the oligonucleotides via conjugation with amine-NHS or thiol-maleimide and conjugated on the other side to ICG via a orthogonal chemistry (e.g., Thiol, maleimide, Amine, Click chemistry). The linker can be a hetero-bifunctional linker that can be attached to the oligonucleotides and the dye with an orthogonal chemistry.

In some embodiments, the functional moiety has been functionalized with an azide group. In further embodiments, the functional moiety is attached to the nucleotide by employing copper-free click chemistry with the azide group. The oligonucleotides are modified with DBCO group and incubated with the azide modified functional moiety.

In some embodiments, orientation of any attached dyes and/or functional moieties is designed. In some embodiments, geometry, orientation, and stoichiometry of any attached dyes and/or functional moieties is designed. In further embodiments, any attached dyes and/or functional moieties face the same direction on the oligonucleotide.

In some embodiments, the nanoparticle may be used as a sensor for bioelectric activity.

In further embodiments, the nanoparticle may be used as a cell voltage sensor.

In some embodiments, the nanoparticle may be used as a photoacoustic probe.

### Nanoparticle Production

Provided is a nanoparticle produced by a process comprising mixing a plurality of DNA oligonucleotides to assemble a DNA scaffold, wherein each oligonucleotide is optionally attached to a dye and/or a functional moiety.

In some embodiments, orientation of any attached dyes and/or functional moieties is designed.

### Methods of Cell Voltage Sensing

Provided is a method of cell voltage sensing at a target site comprising providing the nanoparticle of any one of the preceding embodiments at the target site; and monitoring cell voltage at the target site.

Provided is a method of cell voltage sensing at a target site comprising providing the nanoparticle produced by a process as described herein at the target site; and monitoring cell voltage at the target site.

### Methods of Fluorescence or Absorbance Imaging

Provided is a method of fluorescence imaging at a target site comprising providing the nanoparticle of any one of the preceding embodiments at the target site; and monitoring fluorescence at the target site.

In some embodiments, imaging comprises NIR I, NIR II (also known as short-wave infrared or SWIR) or photoacoustic imaging.

Provided is a method of fluorescence imaging at a target site comprising providing the nanoparticle produced by a process as described herein at the target site; and monitoring fluorescence at the target site.

In some embodiments, imaging comprises NIR I, NIR II or photoacoustic imaging.

Provided is a method of absorbance imaging at a target site comprising providing the nanoparticle of any one of the preceding embodiments at the target site; and monitoring absorbance at the target site.

In some embodiments, imaging comprises NIR I, NIR II or photoacoustic imaging.

Provided is a method of absorbance imaging at a target site comprising providing the nanoparticle produced by a process as described herein at the target site; and monitoring absorbance at the target site.

In some embodiments, imaging comprises NIR I, NIR II or photoacoustic imaging.

### Examples

### Materials and Methods

### Chemicals and Fluorophores

ICG (Catalog #91) and ICG-Xtra-OSu (Catalog #186) were purchased from AAT Bioquest (Pleasanton, CA, USA). FluoVolt Membrane Potential Kit was purchased from Thermo Fisher Scientific (Waltham, MA, USA).

### Oligonucleotides

All oligonucleotides and modified oligonucleotides (cholesterol [5′ Cholesterol-TEG and 3′ Cholesterol-TEG], fluorescently labeled (e.g. FAM [5′ 6-FAM (fluorescein)], TAMRA [3′ 6-TAMRA]), and amino modified [5′ Amino Modifier C6]) used to assemble the DNA scaffolds were purchased from IDT DNA (Coralville, IA, USA) and used without further purification.

### Data Analysis

Raw data acquired from Retiga SensiCam or Kinetix CMOS cameras was converted to 12-bit TIFF images and analyzed in MATLAB_R2022a. Absorbance raw data was exported as a CSV. PA data was recorded and exported by the oscilloscope in the lock-in amplifier as a CSV. All calculations and plots were performed in MATLAB_R2022a or plotted in Microsoft Excel.

### Example 1: DNA Scaffold Design and Assembly

### DNA Scaffold Design

The DNA scaffold was designed using the software Tiamat (41). The sequences of the different strands were designed to be highly orthogonal to maximize folding efficiency and avoid mispairing. The orthogonality of the sequence was validated with NCBI Blast.

DNA Oligonucleotides Conjugation with ICG

Amino modified oligonucleotides were conjugated with ICG fluorophore by incubating them with 5 molar equivalents of ICG-Xtra-OSu in PBS buffer for 2 hours at room temperature and overnight at 4° C. Unreacted dye was removed using isopropanol precipitation. Briefly, the mix was complemented with KOH at 150 mM and 60% isopropanol (ice cold, 99%) and stored in −20° C. for 2 hours prior to 4 hours centrifugation at 4° C. for at least 3 hours (30,000×g). The supernatant was gently removed to avoid discarding the pellet, and the pellet washed two times with ice cold ethanol (99%) via centrifugation (30,000×g, 15 min). The pellet was then dried and resuspended in PBS prior to cleaning by centrifugal filtration (3 kDa MWCO). The efficiency of conjugation was determined using a Nanodrop and fluorescence measurements using a standard curve made with the free ICG dye.

### DIVIN Probes Assembly

To fold the DNA scaffold of the DIVIN probes, 8 short (21-84 nts) ssDNA oligonucleotides (modified or non-modified) were mixed stoichiometrically in UltraPure DNAse/RNAse Free Water (Invitrogen Life Technologies) at a concentration of 125 μM to prepare a stock solution for each construct. Concentrated 10×PBS buffer was then added to form a 1×PBS solution and a final DNA concentration of 60 μM. The buffered DNA solution was then slowly annealed by cooling from 95° C. to 4° C. over 12 hours in a thermocycler (T100 BioRad). The folded probes were used for all experimentation without further purification.

### Example 2: Characterization of DNA Scaffold

### Characterization of Folded DIVIN Probes

DIVIN probes were characterized via Agarose Gel Electrophoresis (AGE) to confirm correct folding of the structure and the absence of bi-products. Folded DIVIN probes were diluted to 2 μM and Gel Loading Dye (B7025S, New England BioTechnologies) was added prior to loading the agarose gel. Agarose gels were formed at 2% and preloaded with 5 g/mL ethidium bromide. Electrophoresis was carried out at 100 V for 30-50 minutes and images were taken using an Azure Gel Imager C150 (Azure Biosystems). To determine the concentration of ICG, absorbance and fluorescence measurements were performed using a Tecan Safire II and a Molecular Devices Spectramax Gemini EM using the following parameters for the wavelengths: Ex. 720 nm And Em. From 770 to 850 nm.

### Fluorescence Resonance Energy Transfer (FRET) Assay for Stability

DIVIN probe were modified with one FRET pair (Fluorescein [FAM]/TAMRA[TAM]). Folded DIVIN probes (500 nM) were incubated in PBS complemented with 20% (v/v) mouse serum to assess their stability. The FRET measurements were performed in a Tecan Safire2 fluorescent plate reader with an excitation at a wavelength set at 455 nm and emission collected from 505 nm to 700 nm. The FRET efficiency and the percentage of intact structures was calculated as in (43).

### Example 3: Surface Plasmon Resonance Assay

Small unilamellar vesicles made of egg-PC (L-α-phosphatidylcholine, Type XVI-E) and prepared using the thin film hydration method were used to create the lipid monolayer. Briefly, the dried Egg-PC lipids were resuspended at a lipid concentration of 5 mg/ml in chloroform in a flat bottom glass vial and subsequently dried in a desiccator for 12 hours under vacuum. The lipid film was then rehydrated using 20 mM HEPES buffered saline solution (pH 7.4) complemented with 150 mM of NaCl. The rehydration was performed at 37° C. for 2 hours and followed by a brief vortexing prior to the extrusion. Liposomes were extruded using a mini extruder (Avanti Polar Lipids, Inc, Alabaster, USA) with two sets of membranes with pore sizes of 200 nm (20 times) and 50 nm (20 times), respectively. The diameter of the liposomes prepared was verified to be around 80 nm using a Nanozetasizer DLS instrument (Malvern Instruments Ltd, Malvern, UK). The liposomes were stored at 4° C. and used for no more than two days. The lipid monolayer was assembled on a hydrophobic MEM gold sensor on the Nicoya OpenSPR instrument. Prior to liposomes loading, the sensor chip wase conditioned with one injection (100 μL) of 40 mM Octyl β-D-glucopyranoside (OGP) solution at a flow rate of 150 μL/min. Liposomes at a concentration of 500 g/mL in HBS buffer were injected at a flow rate 20 μL/min for 5 minutes and left to equilibrate for 10 minutes. The DNA nanoparticles with or without cholesterol moieties were then injected (100 L) over the lipid monolayer at a flow rate of 10 μL/min.

### Example 4: MTT Assay

Cell proliferation was assessed by means of the MTT assay kit (Millipore Sigma, Burlington, USA). HeLa cells were seeded at 1×10³ cell/well in a 96-well plate and incubated in the presence of DIVIN (1 μM, 5 μM, 20 μM) for a period of 24 hours. Next, MTT (10 μL) was added to each sample well, and the plate incubated for 4 hours. Next, 100 μL of solubilization buffer was added to each well, and the plate incubated for 24 hours. The well plate was read using the accuSkan FC microplate photometer (Fisher Scientific, Waltham, USA) at 620 nm. The resulting formazan absorbance measurements were compared against a positive control group treated with PBS and a negative control group treated with ethanol.

### Example 5: Cell Line Electrophysiological Recording and Fluorescence Microscopy

### Cell Line Culture Preparation

HeLa cells were cultured in Eagle's Minimum Essential Media (ATCC Manassas, VA), augmented with 10% Fetal Bovine Serum (VWR, Radnor, USA), 100 IU/mL penicillin and 100 ug/mL streptomycin (Millipore Sigma, Burlington, USA) and were then grown in an incubator at 37° C., 5% CO2 and 90-95% humidity. For electrophysiological experiments, HeLa cells were seeded on standard size glass slides (augmented with Grace Bio-Labs Press-to-Seal silicone isolator) at a concentration of 1.0×10⁵ cells/mL. Electrophysiology experiments were performed 24-48 hours after seeding cells for a cell confluency of ˜30-40%. High concentration DIVIN was briefly sonicated and diluted right before use in PBS at a final concentration of 20 μM. The seeded Hela cells were then incubated with the DIVIN for ˜20-30 minutes at 37° C. The resulting culture was then washed three times with PBS then placed in the patch external solution (42).

### Electrophysiological Recording and Fluorescence Microscopy

Whole-cell patch-clamp recordings were made by ClampX software-controlled patch-clamp amplifier Axopatch (Molecular Devices, San Jose, CA). Glass electrodes filled with patch internal solution (42) were lowered into the Hela cell culture in the patch external solution (42). The patch glass electrodes were pulled from borosilicate glass with a resistance of 5-10μΩ.

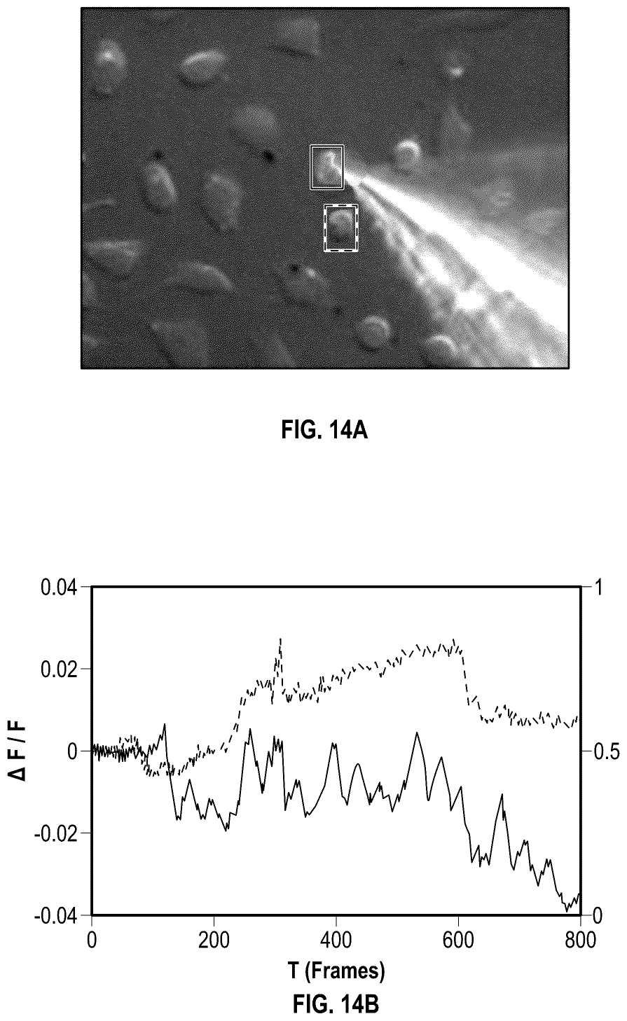

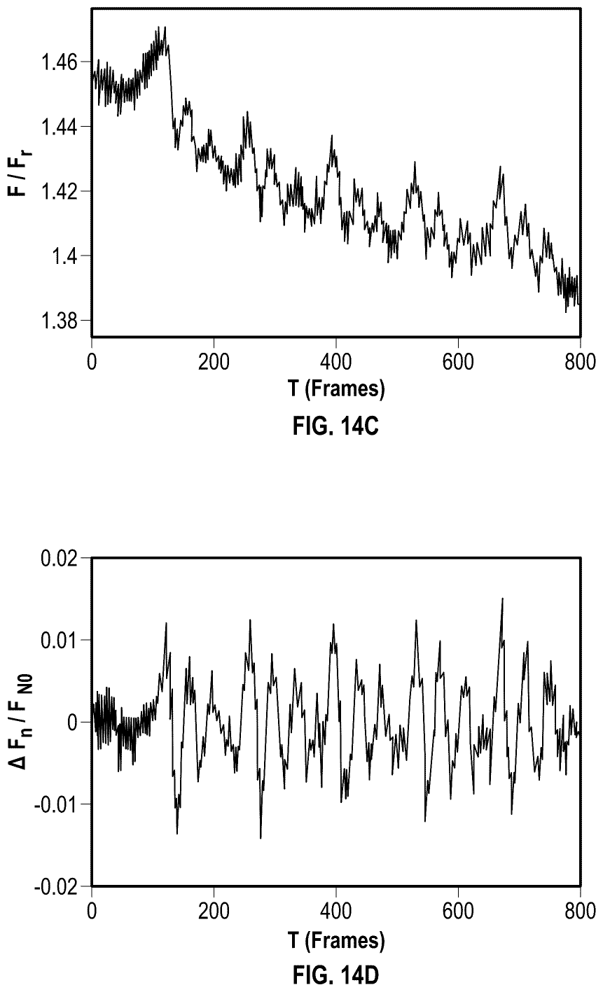

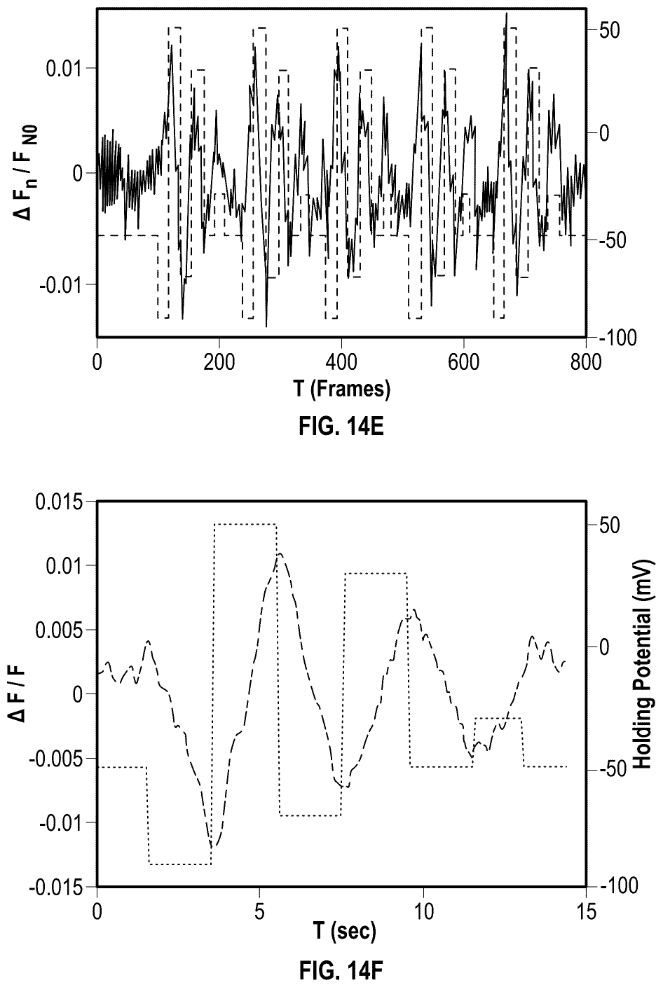

Imaging was done on a custom fluorescence microscope. The filters for DIVIN were bandpass 750 nm-800 nm (excitation), 800 nm longpass (emission), and a 805 nm dichroic mirror. An individual cell was patched and presented a preprogrammed protocol for membrane voltage change. Then one of a number of voltage protocols was used to step through different voltages. In FIG. 13 we present results using the commercially available dye, Fluovolt. Here, the voltage was held at −50 mV before briefly stepping to −70 mV for 0.23 seconds, and then stepping to −90 mV for 2 seconds, +50 mV for 2 seconds, −70 mV for 2 seconds, +30 mV for 2 seconds, −50 mV for 2 seconds, +10 mV for 2 seconds, and −30 mV for 2.23 seconds. Interestingly, in this run the membrane test, a small 5 mV pulse, was turned on at the beginning of the run and appears as a ripple in the fluorescent response. As similar protocol was used in FIG. 14 with the short initial step to −70 mV removed and a return to −50 mV at the end of each sweep. Other protocols started at −50 mV, then alternated between positive and negative values of either 50, 70, or 100 mV every 2 seconds. The fluorescent change was captured by the Retiga SensiCam or Kinetix CMOS camera (Teledyne, Thousand Oaks, USA) and analyzed on the cameras' respective native software.

Fluorescence images of cells stained with DIVIN and DIVIN-Chol were acquired using the aforementioned filters and mirror and Kinetics CMOS camera. Cell cultures were stained with DIVIN and DIVIN-Chol and incubated for 30 minutes just prior to imaging. Images were acquired using varying exposure times typically between 100 and 500 msec and brightness-enhanced to visualize the cells.

The images, then converted into TIFFs, were exported to MATLAB where the following analysis was performed. FIGS. 14A-14F display the typical flow for the analysis. FIG. 14A displays an image of a patched cell stained with DIVIN probes. The continuous line box is a region of interest (ROI) for the patched cell and the dashed line box is an ROI for a non-patched reference cell also stained with DIVIN. Their relative intensities have been displayed in FIG. 14B, where we have normalized them to their initial values for ease of comparison. The next step is to take the target cell's raw intensity and divide it by the reference cell's raw intensity to remove fluctuations in the incoming light, the result is shown in FIG. 14C. This signal was then detrended by removing the slow variations in the signal by applying a high pass filtering the data above a tenth of a Hertz, and the final fractional percentage change for the patched cell is displayed in FIG. 14D. This signal along with the holding potentials are displayed in FIG. 14E of this figure. Finally, the reported fractional fluorescence changes were found by averaging over data from repeated protocols as shown in FIG. 14F. As already discussed, the ICG dye is not as fast as the commercially available Fluovolt is currently not appropriate for signals faster than a fraction of a second as can be seen in the saw-tooth response to the square input.

To further investigate the voltage response of our probes we stained coronal brain slices from a mouse and depolarized the tissue by applying a small volume of osmotically balanced, 300 mM, KCl solution to the dish. Female mice (C57Bl/6) aged 50-100 days were anesthetized using isoflurane and brains were extracted into oxygenated, ice-cold slicing solution (in mM: 2.8 KCl, 10 dextrose, 26.2 NaHCO₃, 1.25 NaH₂PO₄, 0.5 CaCl₂, 7 Mg₂SO₄, and 210 sucrose). Coronal brain slices 350 m in thickness were created using a Leica Vibratome 1000S and were incubated in oxygenated artificial CSF (ACSF; in mM: 126 NaCl, 1.25 NaH₂PO₄, 2.8 KCl, 2 CaCl₂, 1 Mg₂SO₄, 26.2 NaHCO₃, and 11 dextrose) for 40 min at 33° C. and then 40 min at room temperature (24-25° C.). Ingredients for the slicing solution and ACSF were purchased from Fisher Scientific (Hampton, NH, USA). Slices were perfused with 30-32° C. oxygenated ACSF containing 50 μM picrotoxin (Tocris Bioscience, Bristol, UK) at a rate of 2.5 ml/min in a submersion recording chamber. We stained a coronal brain slice from a C57Bl/6 female mouse with 20 μM concentration of DIVIN and depolarized the tissue by applying a small volume of osmotically balanced, 300 mM, KCl solution to the dish.

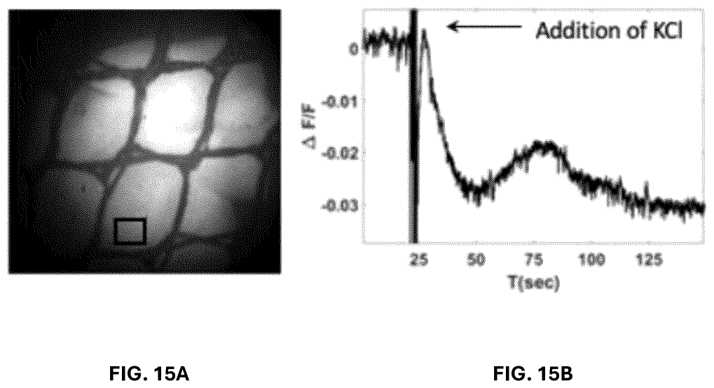

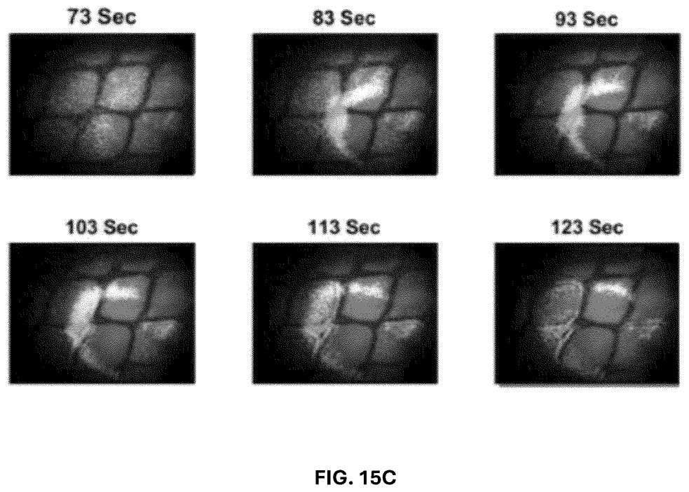

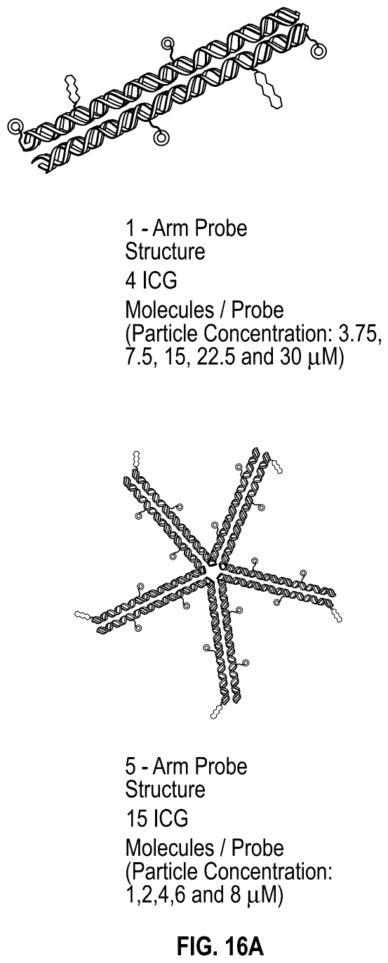

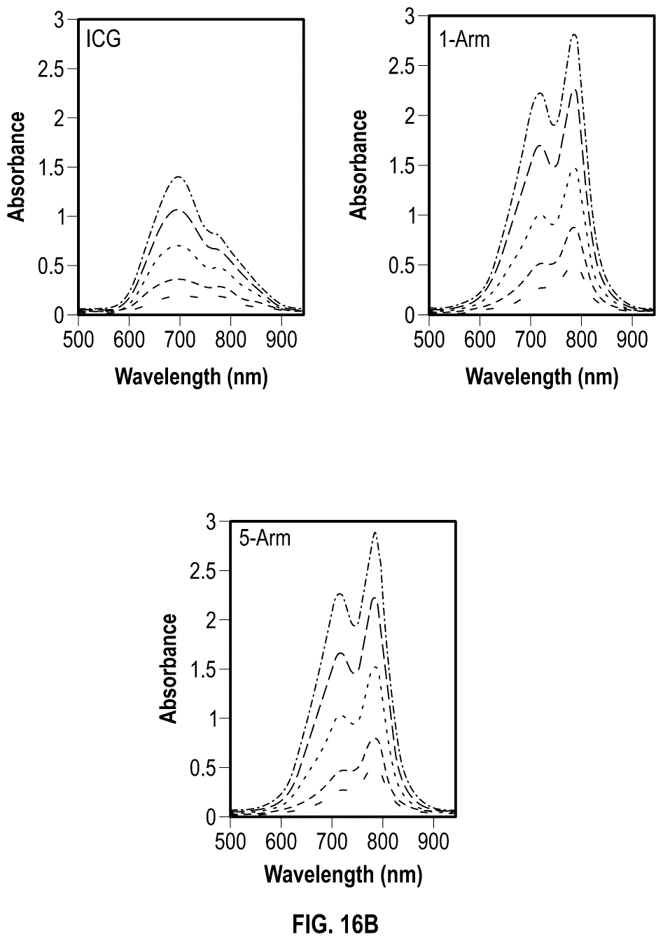

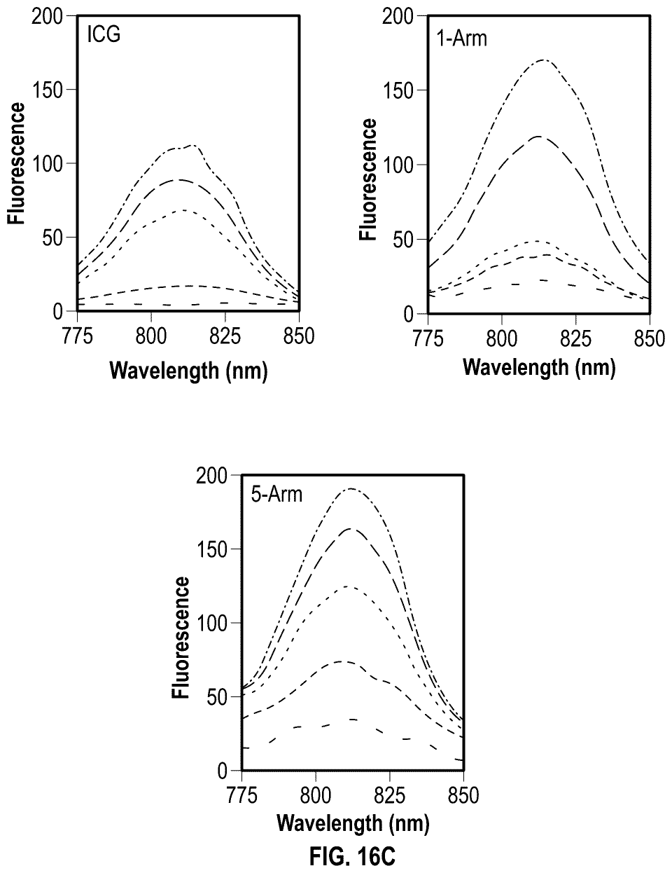

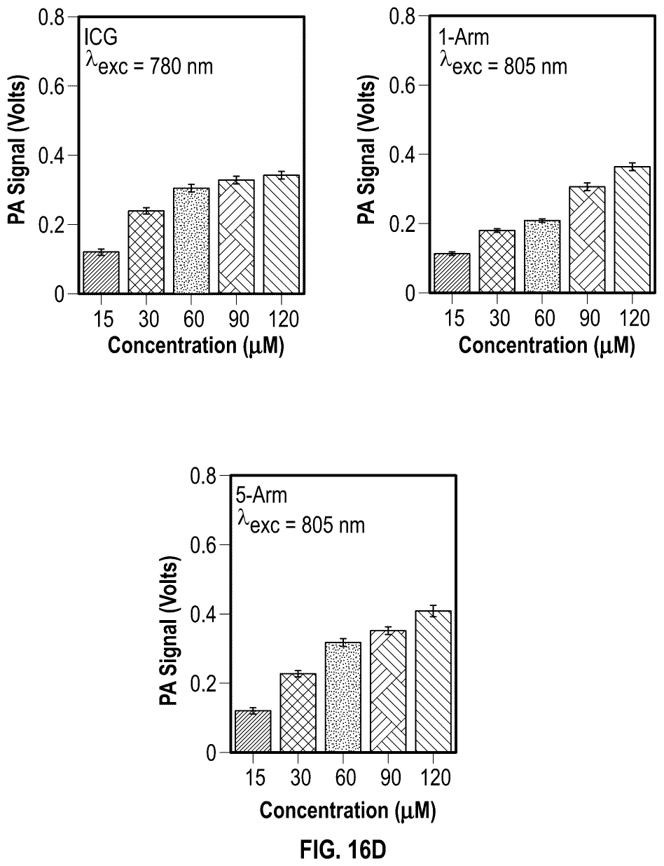

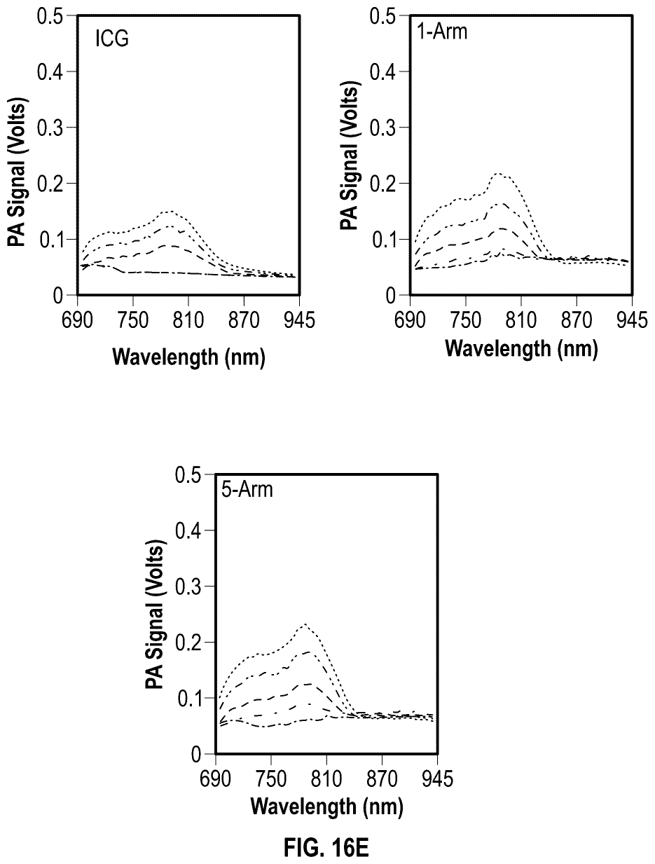

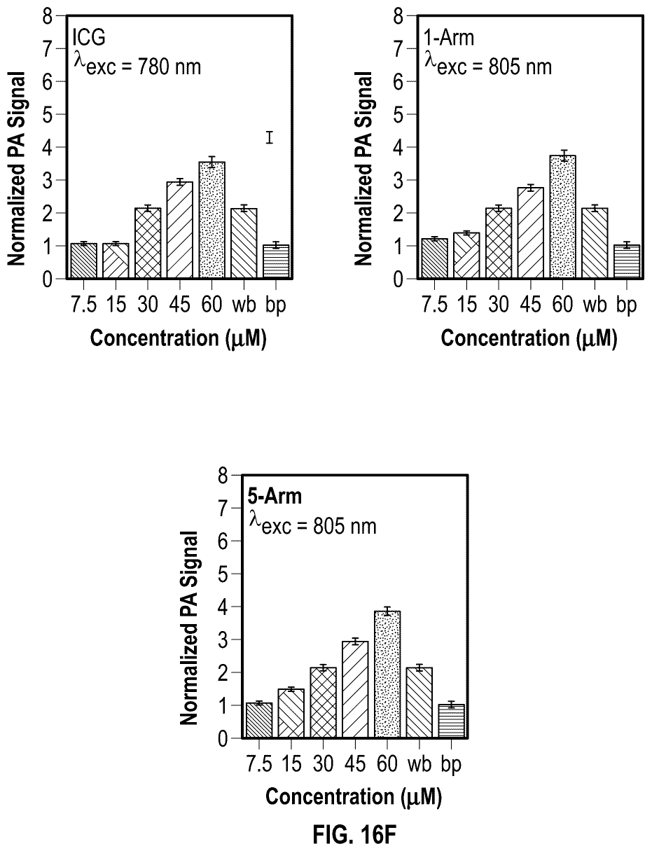

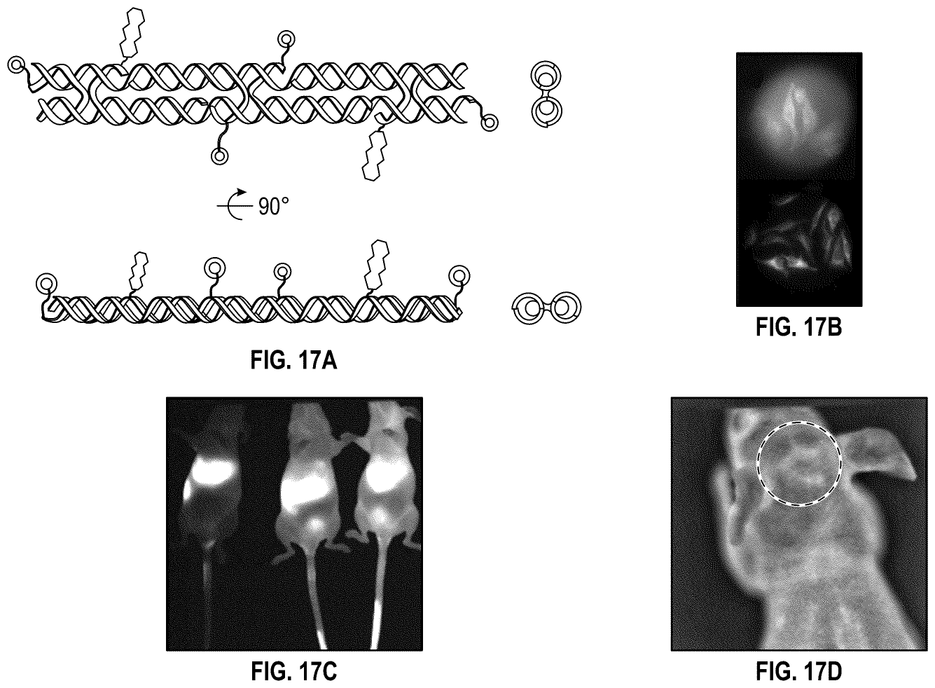

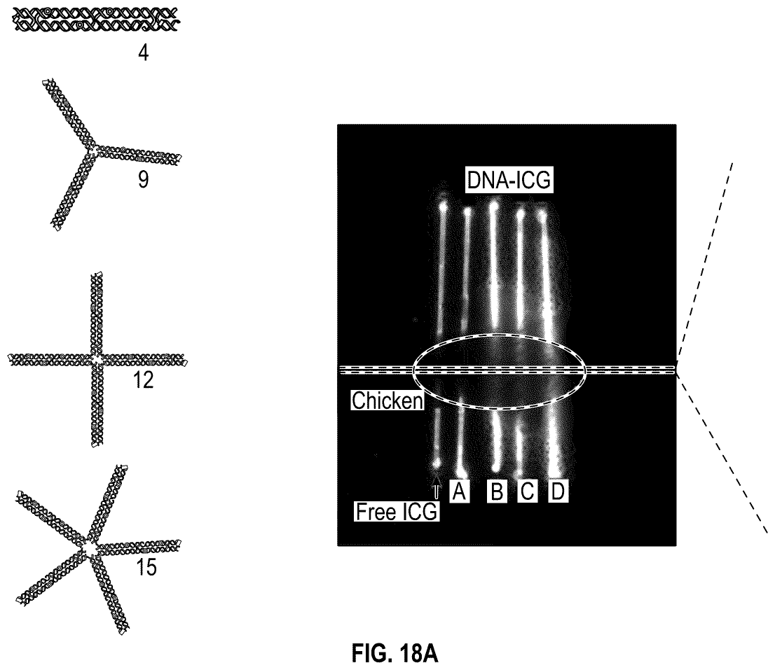

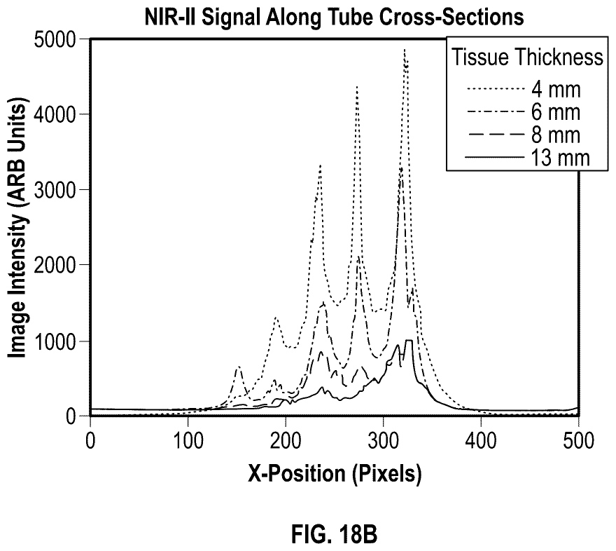

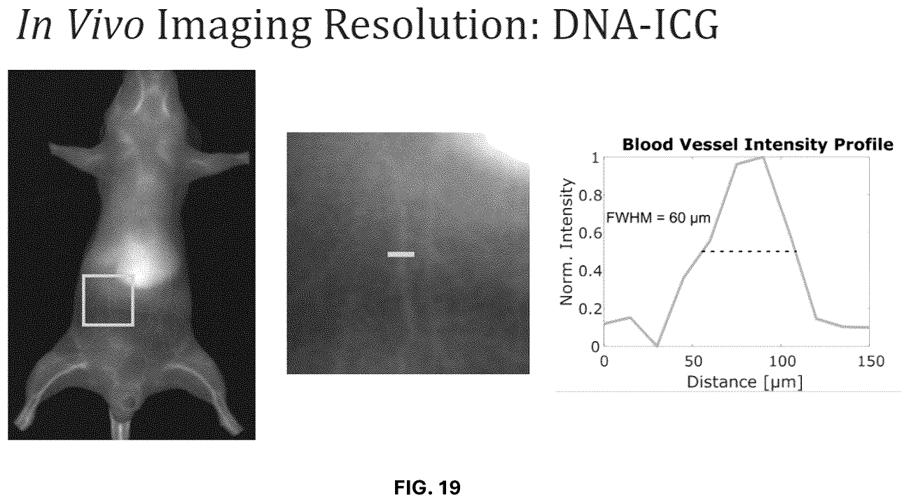

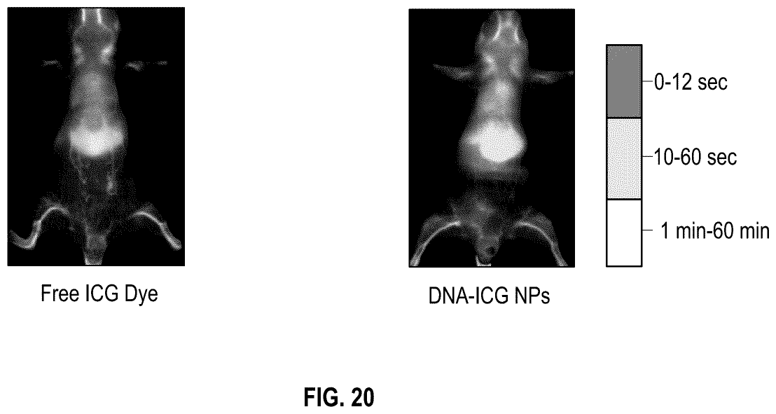

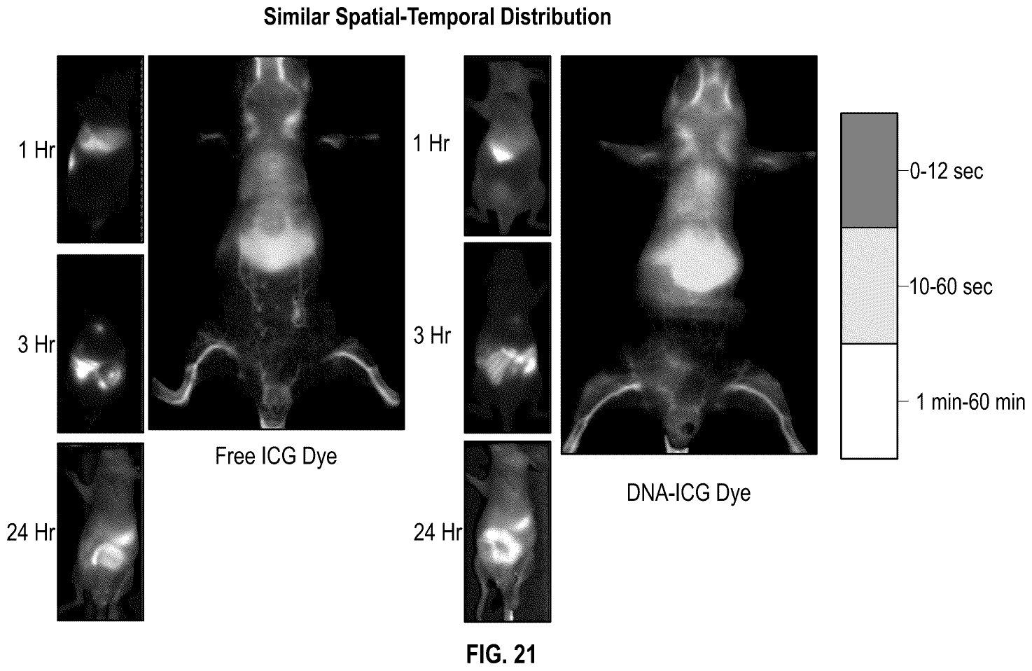

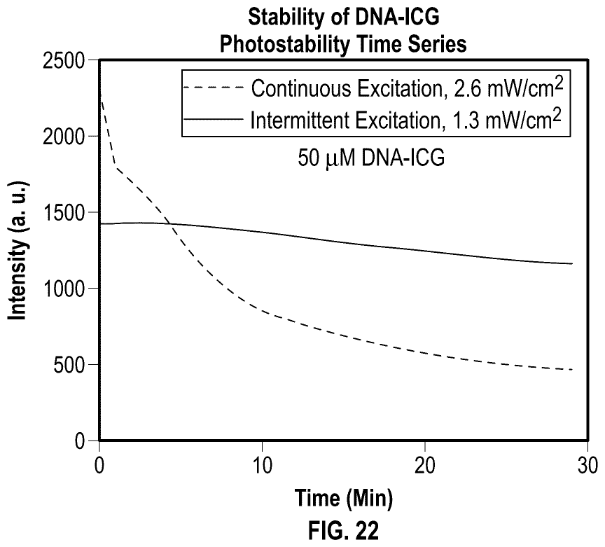

Once the KCl solution has diffused throughout the dish, the final potassium concentration is increased to 30 mM and should result in the near total depolarization of the neuronal and glial cells. The high potassium solution is added to the slice at approximately 25 seconds and results in a three percent decrease in fluorescence. Assuming complete depolarization of the tissue, this fluorescence change results in a 0.04 percent change per mV (FIGS. 15A-15C). This result is slightly higher than that seen in the HeLa cell patch experiments and may be due to better binding of the probes, or potentially greater voltage changes in the normally highly polarized glial network, and spiking activity in the neuronal cells. The wave depicted in the set of images from FIG. 15C which reflects a transient repolarization of the tissue, moves out through the cortex over tens of seconds, which is consistent with the speed of cortical spreading depression (4). These experiments were performed in compliance with George Mason University IACUC protocol 0346.

### Example 6: DIVIN Stability Assay in Whole Blood

The photoacoustic signal of varying concentrations of DIVIN in whole blood was compared in 2-inch segments of 24 gage PTFE tubing (˜100 L). The tubes were filled with various concentrations mixed in whole sheep's blood containing anticoagulant (Hardy Diagnostics, Santa Maria, USA). The tubes were placed in a clear acrylic chamber, which submerged the samples in deionized water. A focused single element piezoelectric 35 MHz transducer was fixed at 12 mm above each sample with the functional element submerged in deionized water. Optimization of positioning and photoacoustic signal from each sample was done through movement of a fine x-y axis stage, moving above each sample with a fixed transducer and pulsed laser. Excitation of the sample was done obliquely by a wavelength-tunable (690-950 nm) pulsed laser designed for photoacoustic imaging (Phocus Mobile, Opotek, Carlsbad, USA) at the peak absorption wavelength for the DIVIN (805 nm) for magnitude comparison. The photoacoustic signal recorded from the transducer is sent to a 20/40 db amplifier (HVA-200M, Femto Somerset, USA) from there, the signal is recorded through a lock-in amplifier (Zurich Instruments, Zurich, Switzerland) triggered on each laser pulse using a photodiode placed near the laser source. For each measurement, a 10 second recording (100 waveforms, 10 Hz) was taken for each sample. For the photostability assay, the sample of DIVIN in whole blood was subjected to 500 shots (50 seconds, 10 Hz) of recording every 15 minutes. The samples were exposed to ˜58 mJ/cm² per 5 nanosecond pulse over the 500 shots every 15 minutes. The absolute amplitude or area under the curve of each signal waveform was calculated, averaged, and normalized to the signal recorded from whole sheep's blood.

### SEQUENCE LISTING

TABLE 1
Non-modified DNA oligonucleotides used to
assemble the DIVIN probe (1-arm).

| | | | SEQ |
|---|---|---|---|
| | | Length | ID |
| Strand | Sequence | (nts) | NO |
| Divin- | TGCAATGTACAGCTGGTCTTGAGCTGATTT | 84 | 1 |
| 1 | AACAAAAGATAACATAACGAGGATACATAT | | |
| | GCGGTGCGGTTGCGATCAACAGCG | | |
| Divin- | CTAGTCTAAGGTGTGGGTTGCAAAGACCAT | 84 | 2 |
| 2 | GTATGAATTAGGAACGAATTCCTTAAAAAA | | |
| | ACTGGTAGACGTACCTAAGAATCT | | |
| Divin- | AGACCAGCGTACGTCTACCAGTTTTTTTAA | 32 | 3 |
| 3 | GG | | |
| Divin- | AATTCGTCGTTATCTTTTGTTAAATCAGCT | 32 | 4 |
| 4 | CA | | |
| Divin- | AACCCACACGCAACCGCACCGCATATGTAT | 31 | 5 |
| 5 | C | | |
| Divin- | CTCGTTATCCTAATTCATACATGGTCTTTG | 31 | 6 |
| 6 | C | | |
| Divin- | CGCTGTTGATCCTTAGACTAG | 21 | 7 |
| 7 | | | |
| Divin- | AGATTCTTAGTGTACATTGCA | 21 | 8 |
| 8 | | | |

TABLE 2
Modified DNA oligonucleotides used to
assemble the DIVIN probe (1-arm).

| | | | SEQ |
|---|---|---|---|
| | | Length | ID |
| Strand | Sequence | (nts) | NO |
| Divin- | AGACCAGCGTACGTCTACCAGTTTT | 32 | 9 |
| 3-ICG | TTTAAGG-ICG | | |
| Divin- | AATTCGTCGTTATCTTTTGTTAAAT | 32 | 10 |
| 4-Chol | CAGCTCA-cholesterol | | |
| Divin- | AACCCACACGCAACCGCACCGCATA | 31 | 11 |
| 5-ICG | TGTATC-ICG | | |
| Divin- | CTCGTTATCCTAATTCATACATGGT | 31 | 12 |
| 6-Chol | CTTTGC-cholesterol | | |
| Divin- | ICG-CGCTGTTGATCCTTAGACTAG | 21 | 13 |
| 7-ICG | | | |
| Divin- | ICG-AGATTCTTAGTGTACATTGCA | 21 | 14 |
| 8-ICG | | | |
| FRET | AACCCACACGCAACCGCACCGCATA | 31 | 15 |
| Divin- | TGTATC-TAM | | |
| 5-TAM | | | |
| FRET | FAM-CCTTAGACTAGTCGCTGTTGA | 22 | 16 |
| Divin- | T | | |
| 7-FAM | | | |

(TAM: TAMRA; FAM: Fluorescein)

TABLE 3
Non-modified DNA oligonucleotides used to
assemble the DIVIN probe (3-arm).

| | | | SEQ |
|---|---|---|---|
| | | Length | ID |
| Strand | Sequence | (nts) | NO |
| 3-arm- | GCTGGTCTTAGTTGGAGGCAATCTACCAGGT | 49 | 17 |
| 1 | CAGACGCGGATACGAGCC | | |
| 3-arm- | CTGGTCTTAGTTGGAAGCTTCCTTAGAGTGC | 47 | 18 |
| 2 | AGGGACGGATACGAGC | | |
| 3-arm- | GCTGGTCTTAGTTGGATGAGACTCGGAAAAT | 49 | 19 |
| 3 | ATTGGTCGGATACGAGCC | | |
| 3-arm- | TAGTAGGACGAAGGCTCGTATCCGTCCAACT | 32 | 20 |
| 4* | A | | |
| 3-arm- | AGACCAGCGTACGTCTACCAGTTTTTTTAAG | 32 | 21 |
| 5* | G | | |
| 3-arm- | TTCGTCCTACTACCTTAAAATGCGGTGCGGT | 51 | 22 |
| 6* | TTGCGTGTGGGTGCAAAGAC | | |
| 3-arm- | CTCGTTATCCTAATTCATACATGGTCTTTGC | 31 | 23 |
| 7* | | | |
| 3-arm- | AACCCACACGCAACCGCACCGCATATGTATC | 31 | 24 |
| 8* | | | |
| 3-arm- | CATGTATGAATTAGGATAACGAGGATACATA | 47 | 25 |
| 9* | AAACTGGTAGACGTAC | | |
| 3-arm- | TCCCTGCACTTTTTCTAAGGAAGCTACCAAT | 75 | 26 |
| 10 | ATTTTTTTTCCGAGTCTCACGTCTGACCTTT | | |
| | TTGGTAGATTGCC | | |

*used 3 times

TABLE 4
Modified DNA oligonucleotides used to
assemble the DIVIN probe (3-arm).

| | | | SEQ |
|---|---|---|---|
| | | Length | ID |
| Strand | Sequence | (nts) | NO |
| 3-arm- | TAGTAGGACGAAGGCTCGTATCCGT | 32 | 27 |
| 4-ICG* | CCAACTA-ICG | | |
| 3-arm- | AGACCAGCGTACGTCTACCAGTTTT | 32 | 28 |
| 5-ICG* | TTTAAGG-ICG | | |
| 3-arm- | AACCCACACGCAACCGCACCGCATA | 31 | 29 |
| 8-ICG* | TGTATC-ICG | | |
| 3-arm- | CTCGTTATCCTAATTCATACATGGT | 31 | 30 |
| 7-Chol* | CTTTGC-cholesterol | | |

*used 3 times

TABLE 5
Non-modified DNA oligonucleotides used to
assemble the DIVIN probe (4-arm).

| | | | SEQ |
|---|---|---|---|
| | | Length | ID |
| Strand | Sequence | (nts) | NO |
| 4-arm- | GCTGGTCTTAGTTGGACATACTTAGAAC | 49 | 31 |
| 1 | ATCATACTTTCGGATCGAGCC | | |
| 4-arm- | CTGGTCTTAGTTGGACCGAAGCGTTGAC | 47 | 32 |
| 2 | TTACTCGCCGGATACGAGC | | |
| 4-arm- | GCTGGTCTTAGTTGGAATCAAACCTAAA | 49 | 33 |
| 3 | TAGTCATATCGGATACGAGCC | | |
| 4-arm- | GCTGGTCTTAGTTGGATCGCTGACTCGC | 49 | 34 |
| 4 | TCTATGCATCGGATACGAGCC | | |
| 4-arm- | TAGTAGGACGAAGGCTCGTATCCGTCCA | 32 | 35 |
| 5* | ACTA | | |
| 4-arm- | AGACCAGCGTACGTCTACCAGTTTTTTT | 32 | 36 |
| 6* | AAGG | | |
| 4-arm- | TTCGTCCTACTACCTTAAAATGCGGTGC | 51 | 37 |
| 7* | GGTTGCGTGTGGGTTGCAAAGAC | | |
| 4-arm- | CTCGTTATCCTAATTCATACATGGTCTT | 31 | 38 |
| 8* | TGC | | |
| 4-arm- | AACCCACACGCAACCGCACCGCATATGT | 31 | 39 |
| 9* | ATC | | |
| 4-arm- | CATGTATGAATTAGGATAACGAGGATAC | 47 | 40 |
| 10* | ATAAAACTGGTAGACGTAC | | |
| 4-arm- | TCCCTGCACTTTTTCTAAGGAAGCTACC | 75 | 41 |
| 11 | AATATTTTGTTTCCGAGTCTCACGTCTG | | |
| | ACCTTTTTGGTAGATTGCC | | |
| 4-arm- | ATGCATAGAGTTTTCGAGTCAGCGAAAA | 50 | 42 |
| 12 | GTATGAGTTTTTTTTAAGTATG | | |

*used 4 times

TABLE 6
Modified DNA oligonucleotides used to
assemble the DIVIN probe (4-arm).

| | | | SEQ |
|---|---|---|---|
| | | Length | ID |
| Strand | Sequence | (nts) | NO |
| 4-arm- | TAGTAGGACGAAGGCTCGTATCCGTC | 32 | 43 |
| 5-ICG* | CAACTA-ICG | | |
| 4-arm- | AGACCAGCGTACGTCTACCAGTTTTT | 32 | 44 |
| 6-ICG* | TTAAGG-ICG | | |
| 4-arm- | AACCCACACGCAACCGCACCGCATAT | 31 | 45 |
| 9-ICG* | GTATC-ICG | | |
| 4-arm- | CTCGTTATCCTAATTCATACATGGTC | 31 | 46 |
| 8-Chol* | TTTGC-cholesterol | | |

*used 4 times

TABLE 7
Non-modified DNA oligonucleotides used to
assemble the DIVIN probe (5-arm).

| | | | SEQ |
|---|---|---|---|
| | | Length | ID |
| Strand | Sequence | (nts) | NO |
| 5-arm- | TCCCTGCACTTTTTCTAAGGAAGCTACC | 75 | 47 |
| 1 | AATATTTTGTTTCCGAGTCTCACGTCTG | | |
| | ACCTTTTTGGTAGATTGCC | | |
| 5-arm- | ATGCATAGAGTTTTCGAGTCAGCGAAAA | 50 | 48 |
| 2 | GTATGAGTTTTTTTTAAGTATG | | |
| 5-arm- | TAGTAGGACGAAGGCTCGTATCCGTCCA | 32 | 49 |
| 3* | ACTA | | |
| 5-arm- | AGACCAGCGTACGTCTACCAGTTTTTTT | 32 | 50 |
| 4* | AAGG | | |
| 5-arm- | TTCGTCCTACTACCTTAAAATGCGGTGC | 51 | 51 |
| 5* | GGTTGCGTGTGGGTTGCAAAGAC | | |
| 5-arm- | CTCGTTATCCTAATTCATACATGGTCTT | 31 | 52 |
| 6* | TGC | | |
| 5-arm- | AACCCACACGCAACCGCACCGCATATGT | 31 | 53 |
| 7* | ATC | | |
| 5-arm- | CATGTATGAATTAGGATAACGAGGATAC | 47 | 54 |
| 8* | ATAAAACTGGTAGACGTAC | | |
| 5-arm- | GCTGGTCTTAGTTGGAGGCAATCTACCA | 49 | 55 |
| 9 | GGTCAGACGCGGATACGAGCC | | |
| 5-arm- | CTGGTCTTAGTTGGATCGCTGACTCGCT | 48 | 56 |
| 10 | CTATGCATCGGATACGAGCC | | |
| 5-arm- | GCTGGTCTTAGTTGGACATACTTAAAAC | 49 | 57 |
| 11 | TCATACTTTCGGATACGAGCC | | |
| 5-arm- | GCTGGTCTTAGTTGGAAGCTTCCTTAGA | 49 | 58 |
| 12 | GTGCAGGGACGGATACGAGCC | | |
| 5-arm- | GCTGGTCTTAGTTGGATGAGACTCGGAA | 49 | 59 |
| 13 | AATATTGGTCGGATACGAGCC | | |

*used 5 times

TABLE 8
Modified DNA oligonucleotides used to
assemble the DIVIN probe (5-arm).

| | | | SEQ |
|---|---|---|---|
| | | Length | ID |
| Strand | Sequence | (nts) | NO |
| 5-arm- | TAGTAGGACGAAGGCTCGTATCCGTCC | 32 | 60 |
| 3-ICG* | AACTA-ICG | | |
| 5-arm- | AGACCAGCGTACGTCTACCAGTTTTTT | 32 | 61 |
| 4-ICG* | TAAGG-ICG | | |
| 5-arm- | AACCCACACGCAACCGCACCGCATATG | 31 | 62 |
| 7-ICG* | TATC-ICG | | |
| 5-arm- | CTCGTTATCCTAATTCATACATGGTCT | 31 | 63 |
| 6-Chol* | TTGC-cholesterol | | |

*used 5 times

### REFERENCES

(1) Swamy, M. M. M.; Murai, Y.; Monde, K.; Tsuboi, S.; Jin, T. Shortwave-Infrared Fluorescent Molecular Imaging Probes Based on π-Conjugation Extended Indocyanine Green. Bioconjugate Chem. 2021, 32 (8), 1541-1547.
(2) Peltrini, R.; Podda, M.; Castiglioni, S.; Di Nuzzo, M. M.; D'Ambra, M.; Lionetti, R.; Sodo, M.; Luglio, G.; Mucilli, F.; Di Saverio, S.; Bracale, U.; Corcione, F. Intraoperative Use of Indocyanine Green Fluorescence Imaging in Rectal Cancer Surgery: The State of the Art. World J Gastroenterol 2021, 27 (38), 6374-6386.
(3) Belia, F.; Biondi, A.; Agnes, A.; Santocchi, P.; Laurino, A.; Lorenzon, L.; Pezzuto, R.; Tirelli, F.; Ferri, L.; D'Ugo, D.; Persiani, R. The Use of Indocyanine Green (ICG) and Near-Infrared (NIR) Fluorescence-Guided Imaging in Gastric Cancer Surgery: A Narrative Review. Front Surg 2022, 9, 880773.
(4) Zhang, Y.; Zhang, Y.; Zhu, J.; Tao, H.; Liang, H.; Chen, Y.; Zhang, Z.; Zhao, J.; Zhang, W. Clinical Application of Indocyanine Green Fluorescence Imaging in Laparoscopic Lymph Node Dissection for Intrahepatic Cholangiocarcinoma: A Pilot Study (with Video). Surgery 2022, 171 (6), 1589-1595.
(5) Liu, Q.; Huang, J.; He, L.; Yang, X.; Yuan, L.; Cheng, D. Molecular Fluorescent Probes for Liver Tumor Imaging. Chemistry—An Asian Journal 2022, 17 (8), e202200091.
(6) Martisiene, I.; Mačianskiene, R.; Treinys, R.; Navalinskas, A.; Almanaitytė, M.; Karčiauskas, D.; Kučinskas, A.; Grigalevičiūtė, R.; Zigmantaitė, V.; Benetis, R.; Jurevičius, J. Voltage-Sensitive Fluorescence of Indocyanine Green in the Heart. Biophysical Journal 2016, 110 (3), 723-732.
(7) Mindt, S.; Karampinis, I.; John, M.; Neumaier, M.; Nowak, K. Stability and Degradation of Indocyanine Green in Plasma, Aqueous Solution and Whole Blood. Photochem Photobiol Sci 2018, 17 (9), 1189-1196.
(8) Mordon, S.; Devoisselle, J. M.; Soulie-Begu, S.; Desmettre, T. Indocyanine Green: Physicochemical Factors Affecting Its Fluorescence in Vivo. Microvascular Research 1998, 55 (2), 146-152.
(9) M. L. Landsman, G. Kwant, G. A. Mook, and W. G. Zijlstra. Light-absorbing properties, stability, and spectral stabilization of indocyanine green. Journal of Applied Physiology 1976, 40 (4), 575-583.
(10) Maarek, J.-M. I.; Holschneider, D. P.; Harimoto, J. Fluorescence of Indocyanine Green in Blood: Intensity Dependence on Concentration and Stabilization with Sodium Polyaspartate. Journal of Photochemistry and Photobiology B: Biology 2001, 65 (2), 157-164.
(11) Mindt, S.; Karampinis, I.; John, M.; Neumaier, M.; Nowak, K. Stability and Degradation of Indocyanine Green in Plasma, Aqueous Solution and Whole Blood. Photochem Photobiol Sci 2018, 17 (9), 1189-1196.
(12) Jaiswal, S.; Dutta, S. B.; Nayak, D.; Gupta, S. Effect of Doxorubicin on the Near-Infrared Optical Properties of Indocyanine Green. ACS Omega 2021, 6 (50), 34842-34849.
(13) Mordon, S.; Devoisselle, J. M.; Soulie-Begu, S.; Desmettre, T. Indocyanine Green: Physicochemical Factors Affecting Its Fluorescence in Vivo. Microvascular Research 1998, 55 (2), 146-152.
(14) Geddes, C. D.; Cao, H.; Lakowicz, J. R. Enhanced Photostability of ICG in Close Proximity to Gold Colloids. Spectrochim Acta A Mol Biomol Spectrosc 2003, 59 (11), 2611-2617.
(15) Williams, S.; Lund, K.; Lin, C.; Wonka, P.; Lindsay, S.; Yan, H. Tiamat: A Three-Dimensional Editing Tool for Complex DNA Structures. In DNA Computing; Goel, A., Simmel, F. C., Sosik, P., Eds.; Lecture Notes in Computer Science; Springer: Berlin, Heidelberg, 2009; pp 90-101.
(16) Ge, Z.; Guo, L.; Wu, G.; Li, J.; Sun, Y.; Hou, Y.; Shi, J.; Song, S.; Wang, L.; Fan, C.; Lu, H.; Li, Q. DNA Origami-Enabled Engineering of Ligand-Drug Conjugates for Targeted Drug Delivery. Small 2020, 16 (16), 1904857.
(17) Veneziano, R.; Ratanalert, S.; Zhang, K.; Zhang, F.; Yan, H.; Chiu, W.; Bathe, M. Designer Nanoscale DNA Assemblies Programmed from the Top Down. Science 2016, 352 (6293), 1534.
(18) Peng, X.; Zhang, Y.; Lu, D.; Guo, Y.; Guo, S. Ultrathin Ti3C2 Nanosheets Based “off-on” Fluorescent Nanoprobe for Rapid and Sensitive Detection of HPV Infection. Sensors and Actuators B: Chemical 2019, 286, 222-229.
(19) Dudani, J. S.; Ibrahim, M.; Kirkpatrick, J.; Warren, A. D.; Bhatia, S. N. Classification of Prostate Cancer Using a Protease Activity Nanosensor Library. Proceedings of the National Academy of Sciences 2018, 115 (36), 8954-8959.
(20) Luo, Y.; Kim, E. H.; Flask, C. A.; Clark, H. A. Nanosensors for the Chemical Imaging of Acetylcholine Using Magnetic Resonance Imaging. ACS Nano 2018, 12 (6), 5761-5773.
(21) Hemmig, E. A.; Fitzgerald, C.; Maffeo, C.; Hecker, L.; Ochmann, S. E.; Aksimentiev, A.; Tinnefeld, P.; Keyser, U. F. Optical Voltage Sensing Using DNA Origami. Nano Lett. 2018, 18 (3), 1962-1971.
(22) Saminathan, A.; Devany, J.; Pillai, K. S.; Veetil, A. T.; Schwake, M.; Krishnan, Y. A DNA-Based Voltmeter for Organelles. bioRxiv 2019, 523019.
(23) Ochmann, S. E.; Joshi, H.; Biiber, E.; Franquelim, H. G.; Stegemann, P.; Sacca, B.; Keyser, U. F.; Aksimentiev, A.; Tinnefeld, P. DNA Origami Voltage Sensors for Transmembrane Potentials with Single-Molecule Sensitivity; 2021; p 2021.08.18.456762.
(24) Weiden, J.; Bastings, M. M. C. DNA Origami Nanostructures for Controlled Therapeutic Drug Delivery. Current Opinion in Colloid & Interface Science 2021, 52, 101411.
(25) Lu, X.; Liu, J.; Wu, X.; Ding, B. Multifunctional DNA Origami Nanoplatforms for Drug Delivery. Chemistry—An Asian Journal 2019, 14 (13), 2193-2202.
(26) Pettersen, E. F.; Goddard, T. D.; Huang, C. C.; Couch, G. S.; Greenblatt, D. M.; Meng, E. C.; Ferrin, T. E. UCSF Chimera—a Visualization System for Exploratory Research and Analysis. J Comput Chem 2004, 25 (13), 1605-1612.
(27) Bui, H.; Díaz, S. A.; Fontana, J.; Chiriboga, M.; Veneziano, R.; Medintz, I. L. Utilizing the Organizational Power of DNA Scaffolds for New Nanophotonic Applications. Advanced Optical Materials 2019, 7 (18), 1900562.
(28) Klais, C. M.; Ober, M. D.; Ciardella, A. P.; Yannuzzi, L. A.; Slakter, J. S. Chapter 53—Diagnostic Indocyanine Green Videoangiography. In Retina (Fourth Edition); Ryan, S. J., Hinton, D. R., Schachat, A. P., Wilkinson, C. P., Eds.; Mosby: Edinburgh, 2006; pp 949-969.
(29) Tsang, V. T. C.; Li, X.; Wong, T. T. W. A Review of Endogenous and Exogenous Contrast Agents Used in Photoacoustic Tomography with Different Sensing Configurations. Sensors (Basel) 2020, 20 (19), 5595.
(30) Xia, J.; Yao, J.; Wang, L. V. Photoacoustic Tomography: Principles and Advances. Electromagn Waves (Camb) 2014, 147, 1-22.
(31) Humbert, J.; Will, O.; Penate-Medina, T.; Penate-Medina, O.; Jansen, O.; Both, M.; Gliier, C.-C. Comparison of Photoacoustic and Fluorescence Tomography for the in Vivo Imaging of ICG-Labelled Liposomes in the Medullary Cavity in Mice. Photoacoustics 2020, 20, 100210.
(32) Yuan, B.; Chen, N.; Zhu, Q. Emission and Absorption Properties of Indocyanine Green in Intralipid Solution. JBO 2004, 9 (3), 497-503.
(33) Qiu, C.; Bai, Y.; Yin, T.; Miao, X.; Gao, R.; Zhou, H.; Ren, J.; Song, L.; Liu, C.; Zheng, H.; Zheng, R. Targeted Imaging of Orthotopic Prostate Cancer by Using Clinical Transformable Photoacoustic Molecular Probe. BMC Cancer 2020, 20 (1), 419.
(34) Macianskiene, R.; Almanaityte, M.; Treinys, R.; Navalinskas, A.; Benetis, R.; Jurevicius, J. Spectral Characteristics of Voltage-Sensitive Indocyanine Green Fluorescence in the Heart. Sci Rep 2017, 7 (1), 7983.
(35) Treger, J. S.; Priest, M. F.; Iezzi, R.; Bezanilla, F. Real-Time Imaging of Electrical Signals with an Infrared FDA-Approved Dye. Biophysical Journal 2014, 107 (6), L09-L12.
(36) Hafid, A.; Difallah, S.; Alves, C.; Abdullah, S.; Folke, M.; Lindén, M.; Kristoffersson, A. State of the Art of Non-Invasive Technologies for Bladder Monitoring: A Scoping Review. Sensors 2023, 23 (5), 2758.
(37) Liu, J.; Zhang, R.; Shang, C.; Zhang, Y.; Feng, Y.; Pan, L.; Xu, B.; Hyeon, T.; Bu, W.; Shi, J.; Du, J. Near-Infrared Voltage Nanosensors Enable Real-Time Imaging of Neuronal Activities in Mice and Zebrafish. J. Am. Chem. Soc. 2020, 142 (17), 7858-7867.
(38) Liu, P.; Miller, E. W. Electrophysiology, Unplugged: Imaging Membrane Potential with Fluorescent Indicators. Acc. Chem. Res. 2020, 53 (1), 11-19.
(39) Tiwari, S.; Sharma, V.; Mujawar, M.; Mishra, Y. K.; Kaushik, A.; Ghosal, A. Biosensors for Epilepsy Management: State-of-Art and Future Aspects. Sensors (Basel) 2019, 19 (7), 1525.
(40) Li, F.; Stewart, C.; Yang, S.; Shi, F.; Cui, W.; Zhang, S.; Wang, H.; Huang, H.; Chen, M.; Han, J. Optical Sensor Array for the Early Diagnosis of Alzheimer's Disease. Front Chem 2022, 10, 874864.
(41) Williams, S.; Lund, K.; Lin, C.; Wonka, P.; Lindsay, S.; Yan, H. Tiamat: A Three-Dimensional Editing Tool for Complex DNA Structures. In DNA Computing; Goel, A., Simmel, F. C., Sosik, P., Eds.; Lecture Notes in Computer Science; Springer: Berlin, Heidelberg, 2009; pp 90-101.
(42) Mathers, D. A.; Leung, W. K.; Fenno, J. C.; Hong, Y.; McBride, B. C. The Major Surface Protein Complex of Treponema Denticola Depolarizes and Induces Ion Channels in HeLa Cell Membranes. Infect Immun 1996, 64 (8), 2904-2910.
(43) Oktay, E.; Alem, F.; Hernandez, K.; Girgis, M.; Green, C.; Mathur, D.; Medintz, I. L.; Narayanan, A.; Veneziano, R. DNA Origami Presenting the Receptor Binding Domain of SARS-CoV-2 Elicit Robust Protective Immune Response. Commun Biol 2023, 6 (1), 1-11.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

What is claimed is:

1. A nanoparticle comprising:
a DNA scaffold comprising an attached dye, an attached functional moiety, or a combination thereof.

2. The nanoparticle of claim 1, wherein the DNA scaffold comprises a plurality of DNA oligonucleotides, wherein each oligonucleotide is independently optionally attached to a dye and/or a functional moiety.

3. The nanoparticle of claim 2, wherein the DNA scaffold comprises one or more oligonucleotides of SEQ ID NO: 1 to 63.

4. The nanoparticle of claim 1, wherein the DNA scaffold comprises one or more arms.

5. The nanoparticle of claim 1, wherein the dye is a cyanine dye.

6. The nanoparticle of claim 1, wherein the dye is selected from the group consisting of indocyanine green (ICG), ICG-Xtra-OSu, IGC-Osu, IGC-Sulfo-Osu, IGC-PEG12-Osu, ICG-NHS, ICG-NH₂, pseudoisocyanine, merocyanine, fluorescein, TAMRA, bis(2,4,6-trihydroxyphenyl)squaraine, tetrtakis(4-sulfonatophenyl)-porphyrin, antimony(III)-phthalocyanine, copper phthalocyanine, perylene bismide, hypericin, subphtalocyanine, heptamethyne cyanine, and combinations thereof.

7. The nanoparticle of claim 1, wherein the functional moiety is selected from the group consisting of a steroid, a cell-penetrating peptide, a targeting peptide, an antibody, or a small molecule.

8. The nanoparticle of claim 1, wherein the dye is attached to the oligonucleotide directly or via a linker.

9. The nanoparticle of claim 1, wherein the functional moiety is attached to the oligonucleotide directly or via a linker.

10. The nanoparticle of claim 9, wherein the linker comprises a polyethylene glycol (PEG) group.

11. The nanoparticle of claim 1, wherein orientation of any attached dyes and/or functional moieties is designed.

12. The nanoparticle of claim 11, wherein any attached dyes and/or functional moieties face the same direction on the oligonucleotide.

13. The nanoparticle of claim 1, wherein the nanoparticle may be used as a sensor for bioelectric activity.

14. The nanoparticle of claim 1, wherein the nanoparticle may be used as a photoacoustic probe.

15. A nanoparticle produced by a process comprising mixing a plurality of DNA oligonucleotides to assemble a DNA scaffold, wherein each oligonucleotide is optionally attached to a dye and/or a functional moiety.

16. The nanoparticle of claim 15, wherein orientation of any attached dyes and/or functional moieties is designed.

17. A method of cell voltage sensing at a target site comprising
providing the nanoparticle of claim 1 at the target site; and
monitoring cell voltage at the target site.

18. A method of cell voltage sensing at a target site comprising
providing the nanoparticle of claim 15 at the target site; and
monitoring cell voltage at the target site.

19. A method of fluorescence imaging at a target site comprising
providing the nanoparticle of claim 1 at the target site; and
monitoring fluorescence at the target site.

20. A method of fluorescence imaging at a target site comprising
providing the nanoparticle of claim 15 at the target site; and
monitoring fluorescence at the target site.

21. A method of absorbance or photoacoustic imaging at a target site comprising
providing the nanoparticle of claim 1 at the target site; and
monitoring absorbance or photoacoustic signal at the target site.

22. A method of absorbance or photoacoustic imaging at a target site comprising
providing the nanoparticle of claim 15 at the target site; and
monitoring absorbance or photoacoustic signal at the target site.


SEQUENCE LISTING (63 sequences)

SEQ ID NO: 1 - DNA, 84 bp, synthetic construct
  source 1..84: mol_type = other DNA
tgcaatgtacagctggtcttgagctgatttaacaaaagataacataacgaggatacatatgcggtgcggttgcgatcaacagcg

SEQ ID NO: 2 - DNA, 84 bp, synthetic construct
  source 1..84: mol_type = other DNA
ctagtctaaggtgtgggttgcaaagaccatgtatgaattaggaacgaattccttaaaaaaactggtagacgtacctaagaatct

SEQ ID NO: 3 - DNA, 32 bp, synthetic construct
  source 1..32: mol_type = other DNA
agaccagcgtacgtctaccagtttttttaagg

SEQ ID NO: 4 - DNA, 32 bp, synthetic construct
  source 1..32: mol_type = other DNA
aattcgtcgttatcttttgttaaatcagctca

SEQ ID NO: 5 - DNA, 31 bp, synthetic construct
  source 1..31: mol_type = other DNA
aacccacacgcaaccgcaccgcatatgtatc

SEQ ID NO: 6 - DNA, 31 bp, synthetic construct
  source 1..31: mol_type = other DNA
ctcgttatcctaattcatacatggtctttgc

SEQ ID NO: 7 - DNA, 21 bp, synthetic construct
  source 1..21: mol_type = other DNA
cgctgttgatccttagactag

SEQ ID NO: 8 - DNA, 21 bp, synthetic construct
  source 1..21: mol_type = other DNA
agattcttagtgtacattgca

SEQ ID NO: 9 - DNA, 32 bp, synthetic construct; identical to SEQ ID NO: 3
  source 1..32: mol_type = other DNA
  misc_feature 32: note = nucleotide conjugated with ICG

SEQ ID NO: 10 - DNA, 32 bp, synthetic construct; identical to SEQ ID NO: 4
  source 1..32: mol_type = other DNA
  misc_feature 32: note = nucleotide conjugated with cholesterol

SEQ ID NO: 11 - DNA, 31 bp, synthetic construct; identical to SEQ ID NO: 5
  source 1..31: mol_type = other DNA
  misc_feature 31: note = nucleotide conjugated wtih ICG

SEQ ID NO: 12 - DNA, 31 bp, synthetic construct; identical to SEQ ID NO: 6
  source 1..31: mol_type = other DNA
  misc_feature 31: note = nucleotide conjugated with cholesterol

SEQ ID NO: 13 - DNA, 21 bp, synthetic construct
  source 1..21: mol_type = other DNA
  misc_feature 1: note = nucleotide conjugated with ICG
cgctgttgatccttagactag

SEQ ID NO: 14 - DNA, 21 bp, synthetic construct
  source 1..21: mol_type = other DNA
  misc_feature 1: note = nucleotide conjugated with ICG
agattcttagtgtacattgca

SEQ ID NO: 15 - DNA, 31 bp, synthetic construct; identical to SEQ ID NO: 5
  source 1..31: mol_type = other DNA
  misc_feature 31: note = nucleotide conjugated with TAMRA

SEQ ID NO: 16 - DNA, 22 bp, synthetic construct
  source 1..22: mol_type = other DNA
  misc_feature 1: note = nucleotide conjugated with fluorescein
ccttagactagtcgctgttgat

SEQ ID NO: 17 - DNA, 49 bp, synthetic construct
  source 1..49: mol_type = other DNA
gctggtcttagttggaggcaatctaccaggtcagacgcggatacgagcc

SEQ ID NO: 18 - DNA, 47 bp, synthetic construct
  source 1..47: mol_type = other DNA
ctggtcttagttggaagcttccttagagtgcagggacggatacgagc

SEQ ID NO: 19 - DNA, 49 bp, synthetic construct
  source 1..49: mol_type = other DNA
gctggtcttagttggatgagactcggaaaatattggtcggatacgagcc

SEQ ID NO: 20 - DNA, 32 bp, synthetic construct
  source 1..32: mol_type = other DNA
tagtaggacgaaggctcgtatccgtccaacta

SEQ ID NO: 21 - DNA, 32 bp, synthetic construct; identical to SEQ ID NO: 3
  source 1..32: mol_type = other DNA

SEQ ID NO: 22 - DNA, 51 bp, synthetic construct
  source 1..51: mol_type = other DNA
ttcgtcctactaccttaaaatgcggtgcggttgcgtgtgggttgcaaagac

SEQ ID NO: 23 - DNA, 31 bp, synthetic construct; identical to SEQ ID NO: 6
  source 1..31: mol_type = other DNA

SEQ ID NO: 24 - DNA, 31 bp, synthetic construct; identical to SEQ ID NO: 5
  source 1..31: mol_type = other DNA

SEQ ID NO: 25 - DNA, 47 bp, synthetic construct
  source 1..47: mol_type = other DNA
catgtatgaattaggataacgaggatacataaaactggtagacgtac

SEQ ID NO: 26 - DNA, 75 bp, synthetic construct
  source 1..75: mol_type = other DNA
tccctgcactttttctaaggaagctaccaatattttttttccgagtctcacgtctgacctttttggtagattgcc

SEQ ID NO: 27 - DNA, 32 bp, synthetic construct; identical to SEQ ID NO: 20
  source 1..32: mol_type = other DNA
  misc_feature 32: note = nucleotide conjugated with ICG

SEQ ID NO: 28 - DNA, 32 bp, synthetic construct; identical to SEQ ID NO: 3
  source 1..32: mol_type = other DNA
  misc_feature 32: note = nucleotide conjugated with ICG

SEQ ID NO: 29 - DNA, 31 bp, synthetic construct; identical to SEQ ID NO: 5
  source 1..31: mol_type = other DNA
  misc_feature 31: note = nucleotide conjugated with ICG

SEQ ID NO: 30 - DNA, 31 bp, synthetic construct; identical to SEQ ID NO: 6
  source 1..31: mol_type = other DNA
  misc_feature 31: note = nucleotide conjugated with cholesterol

SEQ ID NO: 31 - DNA, 49 bp, synthetic construct
  source 1..49: mol_type = other DNA
gctggtcttagttggacatacttagaactcatactttcggatacgagcc

SEQ ID NO: 32 - DNA, 47 bp, synthetic construct
  source 1..47: mol_type = other DNA
ctggtcttagttggaccgaagcgttgacttactcgccggatacgagc

SEQ ID NO: 33 - DNA, 49 bp, synthetic construct
  source 1..49: mol_type = other DNA
gctggtcttagttggaatcaaacctaaatagtcatatcggatacgagcc

SEQ ID NO: 34 - DNA, 49 bp, synthetic construct
  source 1..49: mol_type = other DNA
gctggtcttagttggatcgctgactcgctctatgcatcggatacgagcc

SEQ ID NO: 35 - DNA, 32 bp, synthetic construct; identical to SEQ ID NO: 20
  source 1..32: mol_type = other DNA

SEQ ID NO: 36 - DNA, 32 bp, synthetic construct; identical to SEQ ID NO: 3
  source 1..32: mol_type = other DNA

SEQ ID NO: 37 - DNA, 51 bp, synthetic construct; identical to SEQ ID NO: 22
  source 1..51: mol_type = other DNA

SEQ ID NO: 38 - DNA, 31 bp, synthetic construct; identical to SEQ ID NO: 6
  source 1..31: mol_type = other DNA

SEQ ID NO: 39 - DNA, 31 bp, synthetic construct; identical to SEQ ID NO: 5
  source 1..31: mol_type = other DNA

SEQ ID NO: 40 - DNA, 47 bp, synthetic construct; identical to SEQ ID NO: 25
  source 1..47: mol_type = other DNA

SEQ ID NO: 41 - DNA, 75 bp, synthetic construct
  source 1..75: mol_type = other DNA
tccctgcactttttctaaggaagctaccaatattttgtttccgagtctcacgtctgacctttttggtagattgcc

SEQ ID NO: 42 - DNA, 50 bp, synthetic construct
  source 1..50: mol_type = other DNA
atgcatagagttttcgagtcagcgaaaagtatgagttttttttaagtatg

SEQ ID NO: 43 - DNA, 32 bp, synthetic construct; identical to SEQ ID NO: 20
  source 1..32: mol_type = other DNA
  misc_feature 32: note = nucleotide conjugated wtih ICG

SEQ ID NO: 44 - DNA, 32 bp, synthetic construct; identical to SEQ ID NO: 3
  source 1..32: mol_type = other DNA
  misc_feature 32: note = nucleotide conjugated with ICG

SEQ ID NO: 45 - DNA, 31 bp, synthetic construct; identical to SEQ ID NO: 5
  source 1..31: mol_type = other DNA
  misc_feature 31: note = nucleotide conjugated with ICG

SEQ ID NO: 46 - DNA, 31 bp, synthetic construct; identical to SEQ ID NO: 6
  source 1..31: mol_type = other DNA
  misc_feature 31: note = nucleotide conjugated with cholesterol

SEQ ID NO: 47 - DNA, 75 bp, synthetic construct; identical to SEQ ID NO: 41
  source 1..75: mol_type = other DNA

SEQ ID NO: 48 - DNA, 50 bp, synthetic construct; identical to SEQ ID NO: 42
  source 1..50: mol_type = other DNA

SEQ ID NO: 49 - DNA, 32 bp, synthetic construct; identical to SEQ ID NO: 20
  source 1..32: mol_type = other DNA

SEQ ID NO: 50 - DNA, 32 bp, synthetic construct; identical to SEQ ID NO: 3
  source 1..32: mol_type = other DNA

SEQ ID NO: 51 - DNA, 51 bp, synthetic construct; identical to SEQ ID NO: 22
  source 1..51: mol_type = other DNA

SEQ ID NO: 52 - DNA, 31 bp, synthetic construct; identical to SEQ ID NO: 6
  source 1..31: mol_type = other DNA

SEQ ID NO: 53 - DNA, 31 bp, synthetic construct; identical to SEQ ID NO: 5
  source 1..31: mol_type = other DNA

SEQ ID NO: 54 - DNA, 47 bp, synthetic construct; identical to SEQ ID NO: 25
  source 1..47: mol_type = other DNA

SEQ ID NO: 55 - DNA, 49 bp, synthetic construct; identical to SEQ ID NO: 17
  source 1..49: mol_type = other DNA

SEQ ID NO: 56 - DNA, 48 bp, synthetic construct
  source 1..48: mol_type = other DNA
ctggtcttagttggatcgctgactcgctctatgcatcggatacgagcc

SEQ ID NO: 57 - DNA, 49 bp, synthetic construct
  source 1..49: mol_type = other DNA
gctggtcttagttggacatacttaaaactcatactttcggatacgagcc

SEQ ID NO: 58 - DNA, 49 bp, synthetic construct
  source 1..49: mol_type = other DNA
gctggtcttagttggaagcttccttagagtgcagggacggatacgagcc

SEQ ID NO: 59 - DNA, 49 bp, synthetic construct; identical to SEQ ID NO: 19
  source 1..49: mol_type = other DNA

SEQ ID NO: 60 - DNA, 32 bp, synthetic construct; identical to SEQ ID NO: 20
  source 1..32: mol_type = other DNA
  misc_feature 32: note = nucleotide conjugated with ICG

SEQ ID NO: 61 - DNA, 32 bp, synthetic construct; identical to SEQ ID NO: 3
  source 1..32: mol_type = other DNA
  misc_feature 32: note = nucleotide conjugated with ICG

SEQ ID NO: 62 - DNA, 31 bp, synthetic construct; identical to SEQ ID NO: 5
  source 1..31: mol_type = other DNA
  misc_feature 31: note = nucleotide conjugated with ICG

SEQ ID NO: 63 - DNA, 31 bp, synthetic construct; identical to SEQ ID NO: 6
  source 1..31: mol_type = other DNA
  misc_feature 31: note = nucleotide conjugated with cholesterol